(19) United States
(12) Patent Application Publication
(10) Pub. No.: US 2025/0318745 A1
(43) Pub. Date: Oct. 16, 2025

(54) SYSTEM AND METHOD FOR DIRECT QUANTIFICATION OF PERFUSION METRICS USING A STEPWISE CHANGE IN DEOXYHEMOGLOBIN

(71) Applicant: THORNHILL SCIENTIFIC INC., North York (CA)

(72) Inventors: James DUFFIN, Toronto (CA); Ece Su SAYIN, Windsor (CA); Olivia SOBCZYK, Etobicoke (CA); Julien POUBLANC, North York (CA); David J. MIKULIS, Oakville (CA); Joseph Arnold FISHER, Thornhill (CA)

(21) Appl. No.: 19/249,790

(22) Filed: Jun. 25, 2025

Related U.S. Application Data
(63) Continuation-in-part of application No. 18/841,113, filed on Aug. 23, 2024.
(60) Provisional application No. 63/313,996, filed on Feb. 25, 2022.
(60) Provisional application No. 63/663,945, filed on Jun. 25, 2024.

(51) Int. Cl. A61B 5/055; A61B 5/00; A61B 5/02; A61B 5/026

(52) U.S. Cl. CPC A61B 5/055; A61B 5/02028; A61B 5/0263; A61B 5/7271

(57) ABSTRACT

An improved method and system are provided for determining a perfusion metric in a subject using magnetic resonance imaging and physiologically induced contrast. A respiratory device induces a stepwise increase in arterial partial pressure of oxygen by sequentially delivering hypoxic and oxygenated gas mixtures. Magnetic resonance signal data are acquired from a selected voxel, and a change in effective transverse relaxation rate (ΔR₂*) is computed over time. A perfusion metric is determined based on the ΔR₂* time course without requiring deconvolution of an arterial input function. In some examples, the ΔR₂* response is characterized using a sigmoidal model such as a Gompertz function to extract physiologically relevant parameters including relative cerebral blood volume, relative cerebral blood flow, and mean transit time. A processor may compute perfusion metrics across multiple voxels and output a perfusion map or compare values to a reference population to assess tissue abnormality for diagnostic or treatment purposes.

### CROSS-REFERENCE TO RELATED APPLICATION(S)

This application is a continuation-in-part of 18/841113 entitled “DYNAMIC SUSCEPTIBILITY CONTRAST USING A PRE-DETERMINED ARTERIAL INPUT FUNCTION”, filed Aug. 23, 2024, which is a 371 of PCT International Patent Application PCT/IB2023/051818, filed Feb. 27, 2023, which claims priority to U.S. Provisional Patent Application No. 63/313,996, entitled “The use of the end-tidal PO2 (P_{ET}O₂) as the AIF” filed Feb. 25, 2022, and this application also claims priority to U.S. Provisional Patent Application No. 63/663,945, filed Jun. 25, 2024 the entire contents of which are incorporated herein by reference.

### FIELD

The present specification is directed to perfusion MRI, and specifically methods and systems for using deoxyhemoglobin as a contrast agent.

### BACKGROUND

Many common conditions such as cigarette smoking, high blood cholesterol, obesity, sedentary lifestyle, diabetes, hypertension, and aging result in silently accumulating cerebrovascular pathologies, for example small vessel disease, venous collagenases, chronic inflammation and multiple subcortical infarcts. The health of cerebral perfusion can be assessed by perfusion metrics calculated using dynamic susceptibility contrast (DSC).

A considerable source of error in the calculation of perfusion metrics is the uncertainty of the arterial input function (AIF). To determine the AIF, a magnetic resonance imaging (MRI) signal is typically measured over a large artery such as the middle cerebral artery (MCA) while implementing a bolus of contrast agent. Unfortunately, this method of determining the AIF is not practical when the MCA is small, or oriented in a direction not suitable to measure the AIF, or in organs that lack sufficiently large arteries, for example, the thyroid gland. Failing that, no AIF can be identified, precluding calculation of hemodynamic metrics. Furthermore, with respect to calculating deoxyhemoglobin from end-tidal breath values as a contrast agent, the temporal resolution for the AIF is limited to the respiratory rate, which may be significantly longer than the TR of the MRI system.

### SUMMARY

The poor temporal-resolution and averaging limitations that arise when perfusion is calculated from an arterial-input function are addressed by inducing a controlled step-wise reoxygenation in the subject and analyzing the resulting voxel signal directly. The specification therefore provides a workflow that yields quantitative perfusion metrics with improved fidelity while obviating selection of an arterial reference.

An aspect of the specification provides a method of determining at least one perfusion metric in a subject. The method includes inducing a step-wise increase in arterial partial pressure of oxygen by first delivering a hypoxic gas to create arterial hypoxia and then delivering an oxygenated gas to re-oxygenate the arterial blood. A magnetic resonance imaging system acquires a magnetic signal from a selected voxel and produces a time-course of the change in effective transverse relaxation rate that results from the oxygenation step. At least one perfusion metric for that voxel is then determined from the time-course.

In one example, the hypoxic gas is supplied over a series of tidal breaths and the oxygenated gas is supplied in a single breath.

In another example, a sequential gas-delivery apparatus targets a first end-tidal partial pressure of oxygen during the hypoxic phase and a higher second end-tidal partial pressure of oxygen during reoxygenation while maintaining end-tidal partial pressure of carbon dioxide.

In a further example, the first end-tidal partial pressure of oxygen is about 40 mmHg and the second end-tidal partial pressure of oxygen is about 95 mmHg.

In one example, the voxel time-course is fitted with a predetermined sigmoid function, and the perfusion metric is determined from parameters of the pre-determined function.

In another example, the predetermined sigmoid function is a Gompertz function expressed as

$${S}_{fit} ( t ) = {S}_{base} + a {e}^{- {be}^{- ct}}$$

$$wherein :$$

$$S = Δ {R}_{2}^{*} ;$$

$$t = time ;$$

$${S}_{fit ( t )} = the fitted Δ {R}_{2}^{*} signal time course of the step response ;$$

$${S}_{base} = the initial value of {S}_{fit ( t )} ;$$

$$a = the magnitude of the S decrease ;$$

$$b = the displacement along the time axis ;$$

$$and$$

$$c = the rate of change .$$

In one example, the hemoglobin concentration is assumed to be about 130 g L-1 in healthy women and about 150 g L⁻¹ in healthy men.

In another example, the blood pH is assumed to be about 7.4.

In one example, the perfusion metric includes relative cerebral blood volume and is determined from the magnitude of the predetermined sigmoid function.

In another example, the perfusion metric includes relative cerebral blood flow and is determined from the maximum rate of decrease of the predetermined sigmoid function.

In a further example, the perfusion metric includes mean transit time, which is calculated as the ratio of relative cerebral blood volume to relative cerebral blood flow.

In one example, perfusion metrics are determined for a plurality of voxels, co-registered to an anatomical image, and displayed as a perfusion map.

In another example, a perfusion metric for a voxel is compared with a statistical value representing the same metric in corresponding voxels of a reference population, and a z-score is generated.

In one example, a health condition is assessed or diagnosed from the z-score.

In another example, the health condition corresponds to one or more of Parkinson's disease, stroke, hemangioma, vascular tumor, coronary heart disease, Moyamoya disease, cerebral venous thrombosis, arteriovenous malformation, arterio-venous fistula, angioma formation, carotid artery disease, intracranial hypertension, steno-occlusive disease, or kidney insufficiency.

In a further example, effectiveness of a treatment is assessed from the z-score.

A further aspect of the specification provides a system for quantifying a perfusion metric in a subject. The system includes a respiratory device that induces the step-wise arterial oxygen increase by delivering hypoxic gas followed by oxygenated gas, a magnetic resonance imaging device that acquires a voxel signal and produces the corresponding change in relaxation-rate time-course, and at least one processor that determines at least one perfusion metric for the voxel from that time-course.

In one example, the system processor determines perfusion metrics for multiple voxels, co-registers the metrics to an anatomical image, and generates a perfusion map.

In another example, the processor compares a voxel perfusion metric with a statistical reference value, generates a z-score, and assesses or diagnoses a health condition from that z-score.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

The patent or application file contains at least one drawing executed in color. Copies of this patent or patent application publication with color drawings will be provided by the Office upon request and payment of the necessary fee.

The present disclosure will be described with respect to the following figures:

FIG. 1 is a graph of a stepwise change in the concentration of deoxyhemoglobin in the lung, according to one embodiment.

FIG. 2 is a schematic representation of the trumpet model for a lung.

FIG. 3A is a graph of a compartment model for a voxel.

FIG. 3B is a graph of capillary population model for a voxel.

FIG. 4 is a graph of a wavefront model for a voxel.

FIG. 5 is a schematic diagram of a system for determining perfusion metrics according to one embodiment.

FIG. 6 is a schematic diagram of a method of determining perfusion metrics using the system of FIG. 5, according to one embodiment.

FIG. 7 is a graph representing exemplary performance of the method of FIG. 6.

FIG. 8A is a graph of the ΔR₂* signal measured during exemplary performance of the method of FIG. 6.

FIG. 8B is a graph of the ΔR₂* signal measured during exemplary performance of the method of FIG. 6.

FIG. 9 is a graph of the perfusion metrics computed for gray matter during exemplary performance of the method of FIG. 6.

FIG. 10 is a graph of the perfusion metrics computed for white matter during exemplary performance of the method of FIG. 6 and compared to the prior art methods.

FIG. 11 is a graph of the perfusion metrics computed during exemplary performance of the method of FIG. 6 and compared to the prior art methods.

FIG. 12 is a graph of the perfusion metrics computed during exemplary performance of the method of FIG. 6 and compared to the prior art methods.

FIG. 13 is a graph of the perfusion metrics computed during exemplary performance of the method of FIG. 6 and compared to the prior art methods.

FIG. 14 is a perfusion map generated during exemplary performance of the method of FIG. 6 and compared to the prior art methods.

FIG. 15 is a perfusion map generated during exemplary performance of the method of FIG. 6 and compared to the prior art methods.

FIG. 16 is a perfusion map generated during exemplary performance of the method of FIG. 6 and compared to the prior art methods.

FIG. 17 is a perfusion map generated during exemplary performance of the method of FIG. 5 and compared to the prior art methods.

FIG. 18 is a perfusion map generated during exemplary performance of the method of FIG. 6 and compared to the prior art methods.

FIG. 19 is a graph of the magnetic signal measured during exemplary performance of the method of FIG. 6.

FIG. 20A is a graph of the ΔR₂* signal measured during exemplary performance of the method of FIG. 6.

FIG. 20B is a graph of the ΔR₂* signal measured during exemplary performance of the method of FIG. 6.

### DETAILED DESCRIPTION

The following abbreviations are used herein:

| | |
|---|---|
| AIF | arterial input function |
| ANOVA | analysis of variance |
| a.u. | arbitrary units |
| BOLD | blood oxygen level dependent imaging |
| CSF | cerebral spinal fluid |
| G1 | first gas |
| G2 | second gas |
| GM | gray matter |
| FRC | functional residual capacity |
| MCA | middle cerebral artery |
| MRI | magnetic resonance imaging |
| MTT | mean transit time |
| GBCA | gadolinium-based contrast agents |
| PₐCO₂ | arterial partial pressure of carbon dioxide |
| PₐO₂ | arterial partial pressure of oxygen |
| PCO₂ | partial pressure of carbon dioxide |
| PO₂ | partial pressure of oxygen |
| P_{ET}CO₂ | end tidal partial pressure of carbon dioxide |
| P_{ET}O₂ | end tidal partial pressure of oxygen |
| rBAT | relative blood arrival time |
| rCBF | relative cerebral blood flow |
| rCBV | relative cerebral blood volume |
| ΔR₂* | change in the effective transverse relaxation rate (inverse of the |
| | T2* signal) |
| S | ΔR₂* signal in a voxel |
| SaO₂ | arterial blood-oxygen saturation |
| TE | echo time |
| THx-dOHb | transient hypoxia-induced deoxyhemoglobin |
| THx-dOHb-AIF | transient hypoxia-induced deoxyhemoglobin analyzed using an |
| | arterial input function |
| THx-dOHb-Step | transient hypoxia-induced deoxyhemoglobin analyzed using the |
| | step reoxygenation (recovery) |
| TR | repetition time |
| WM | white matter |

The following definitions are used herein:

“About” herein refers to a range of +20% of the numerical value that follows. In one example, the term “about” refers to a range of +10% of the numerical value that follows. In another example, the term “about” refers to a range of +5% of the numerical value that follows.

“Hypoxic” herein refers to blood with abnormally low oxygen levels. Generally, a hypoxic PₐO₂ is below about 80 mmHg.

“Normoxic” herein refers to blood with normal oxygen levels. Generally, a normoxic PₐO₂ is between about 70 mmHg and about 110 mmHg.

Resting cerebral perfusion metrics can be calculated from the MRI ΔR₂* signal during the first passage of an intravascular bolus of a Gadolinium-based contrast agent (GBCA), or more recently, a transient hypoxia-induced change in the concentration of deoxyhemoglobin ([dOHb]). Conventional analysis calculates the concentration of the contrast agent in the voxel via deconvolution of the tissue signal with an arterial input function (AIF). Typically, the sharpest signal change identified over the middle cerebral artery or choroid plexus is assumed to be the AIF for use in a deconvolution-based kinetic model to calculate mean transit time (MTT) and relative cerebral blood volume (rCBV). The proxy process encompasses errors inherent in designating an AIF and performing deconvolution calculations.

An improved method of computing perfusion metrics is provided. The method is premised on a direct analysis model, as described herein.

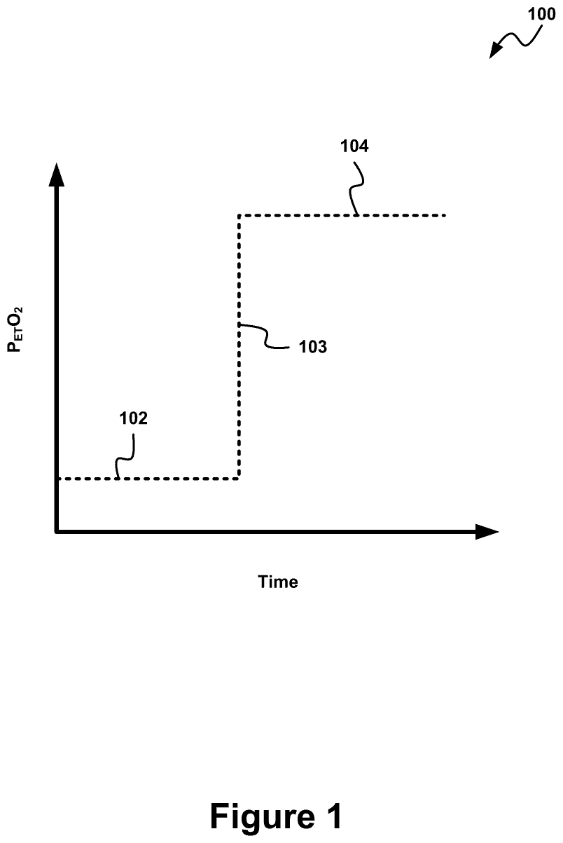

The perfusion metric can be induced by implementing a stepwise change in deoxyhemoglobin in the subject. FIG. 1 is a graph 100 displaying time on the x-axis and the subject's P_{ET}O₂ on the y-axis. As indicated at 202, hypoxia is targeted in the subject 330 using a respiratory device, then the subject's lung is abruptly reoxygenated (indicated at 104).

### Trumpet (or Thumbtack) Model

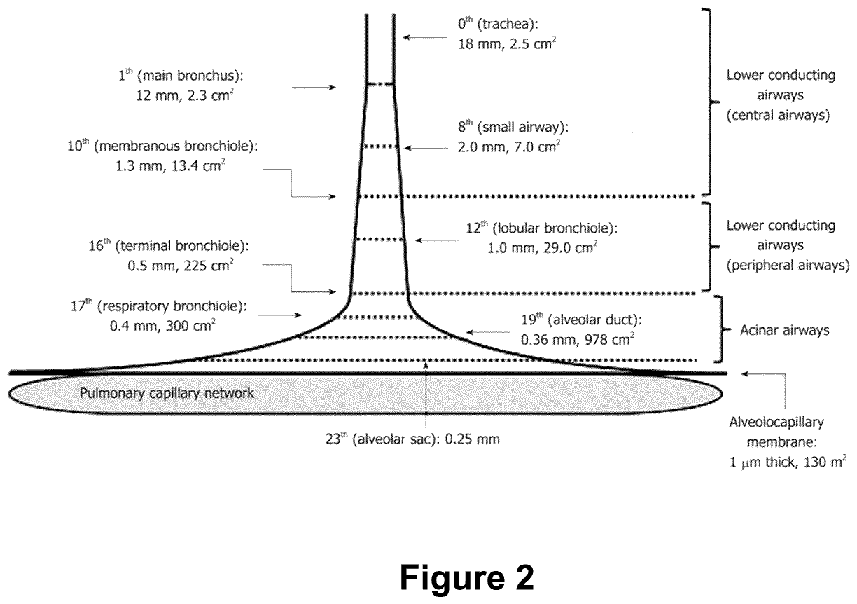

FIG. 2 provides a schematic representation of the effective diameter and cross-sectional area along lower conducting airways and acinar airways of the lung. In the reoxygenation phase, there is a remarkable anatomical-physiological feature that enables full saturation of the pulmonary venous blood in a fraction of a second. During an inspiration, inhaled gas passes down successive generations of branches of bronchi and alveolar ducts while undergoing minimal gas exchange. But, in the fraction of a second that the inspired gas passes the 16th branch of airways into the alveolae where most of the gas exchange takes place, the surface area for oxygen diffusion expands from 225 cm² to 130 m², resulting in a near instantaneous change in the oxygen partial pressure of the pulmonary capillary blood.

Breathing a suitably hypoxic gas can maintain pulmonary venous, and thus arterial PO2 at, for example, 40 mmHg, equivalent to an SaO₂ about 75%. During a single inspiration of oxygen-enriched air, the pulmonary alveoli, and thus alveolar capillary blood, undergo abrupt oxygen saturation. The blood which has suddenly increased its SaO₂ is conducted into the pulmonary vein, left atrium and ventricle, and enters the arterial tree, retaining the same abrupt leading edge of hemoglobin saturation at every branching as the vessels ramify into the brain. This rapid transition from deoxyhemoglobin to oxyhemoglobin describes a susceptibility contrast agent step function.

The simplifying assumption for the analysis of step changes in [dOHb](THx-dOHb-Step) is that the ΔR₂* signal(S) in a voxel is directly proportional to [dOHb] or (1-SaO₂). Equation 1 sets out this proportionality. co-registered to anatomical images.

$$S = C \times [ Hb ] \times ( 1 - {SaO}_{2} ) \times CBV \qquad ( 1 )$$

In Equation 2, S is the ΔR₂* signal in a voxel; C is a proportionality constant; CBV, the volume of blood in a voxel; SaO₂ is the arterial oxygen saturation; [Hb] is the arterial hemoglobin concentration (assumed to be 130 g/L unless measured). SaO₂ is related to arterial PO2 (PₐO₂) by the in vivo oxygen dissociation curve. Equation 2 describes the relation:

$${SaO}_{2} = \frac{K \times {PO}_{2}^{n}}{( 1 + K \times {PO}_{2}^{n} )} \qquad ( 2 )$$

In Equation 2, n and K are derived from a Levenburg-Marquardt fit to measured human data. K=5×10^{-142x}(pH)^{157.31} and n=−4.4921×pH+36.365. pH is assumed to be 7.4 unless measured.

### Current Models for Voxel-Wise Analysis of Hemodynamics

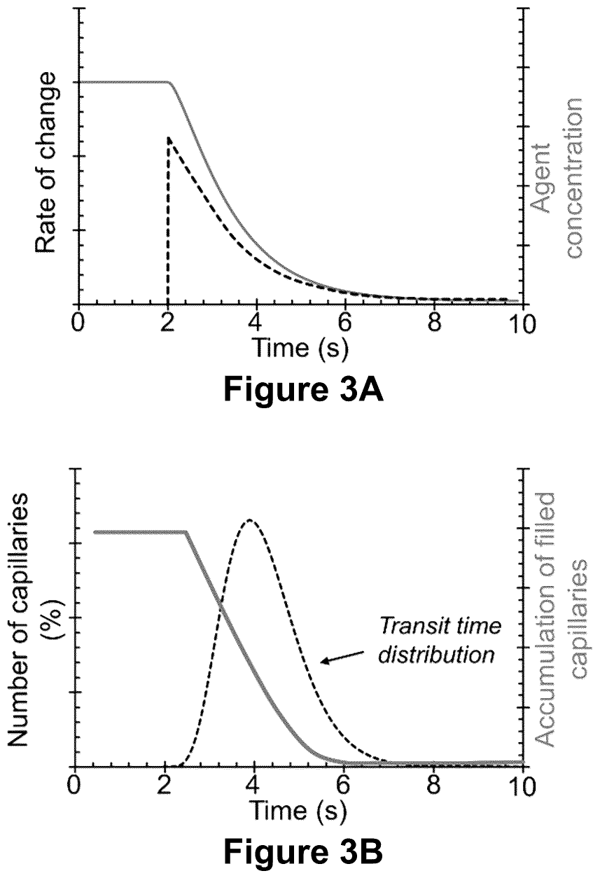

The two models commonly used to describe the process of displacement of one indicator with another are illustrated in FIGS. 3A and 3B. FIG. 3A is a graph modelling instantaneous homogeneity. According to this model, blood with decreased contrast agent enters a well-mixed container, so that the initial high rate of decrease of [dOHb] is a declining exponential (dashed line), approaching an asymptote (solid line). FIG. 3B is a graph modelling the population of capillaries. According to this model, blood with decreased contrast agent [dOHb] enters the voxel capillary population simultaneously, initially perfusing all capillaries, producing a linear decline in net voxel [dOHb](red line) proportional to the blood flow into the voxel. Capillaries with progressively longer transit times continue to fill with oxygenated blood, resulting in an exponential decline in the net [dOHb] to an asymptote (solid line).

If the voxel is viewed as a compartment, the contents of which undergo instantaneous mixing with the inflowing indicator as illustrated in FIG. 3A, the signal intensity response to a step change in susceptibility contrast agent such as dOHb reflects the balance of contrast agent in the course of the exchange. In the case of a high [dOHb] in the voxel and a low [dOHb] in the inflow, the initial high rate of signal intensity decline falls off exponentially as the difference in inflow and outflow of contrast declines to zero. The time constant of this exponential is MTT. Thus, model FIG. 3A describes the central volume theorem where MTT=CBV/CBF. This is a generic kinetic model for unknown patterns of blood flow through the tissues and is applied for the calculation of perfusion metrics using the prior art methods, described in the examples as GBCA-AIF and THx-dOHb-AIF.

Alternatively, if the displacement of the high susceptibility contrast such as dOHb in the voxel is viewed as a filling of a population of capillaries with varying transit times, then the rate of decrease of the signal intensity response is attributed to the distribution of the blood with reduced [dOHb] to an assumed bundle of capillaries having a normal distribution of blood transit times, as illustrated in FIG. 3B. As blood with decreased [dOHb] simultaneously enters the voxel's population of capillaries, the signal intensity decreases linearly until capillaries with shortest transit times are filled with the decreased contrast agent. Thereafter, the rate of decline of the signal intensity slows as oxygenated blood progressively replaces deoxygenated blood in the voxel.

### Direct Analysis Model

The prior art analyses (GBCA AIF and THx-dOHb-AIF) use the kinetic model with first order dynamics shown in FIG. 3A. In that case, the residue function is an exponential curve with a time constant MTT as stated in Equation 4 (described herein). However, it is unlikely this simple model describes the physiological events sufficiently to be the basis for the analysis of the ΔR₂* signal response to a step decrease in [dOHb]. Indeed, in examining the step response patterns of the ΔR₂* signal during re-oxygenation in multiple voxels with little noise, despite generating a near instantaneous initial step change in susceptibility contrast agent proximally in the major arteries, at the voxel level, most voxels had a period of initial acceleration of signal decline. This was followed by a period of linear decrease, ending with a period where the rate of decline decelerated to zero. Note that the models shown in FIGS. 2A and 2B have their maximal rate of decline at the beginning. The model shown in FIG. 2B begins with a sudden transition from zero change in contrast agent, to a constant rate of decline, decelerating to a steady value.

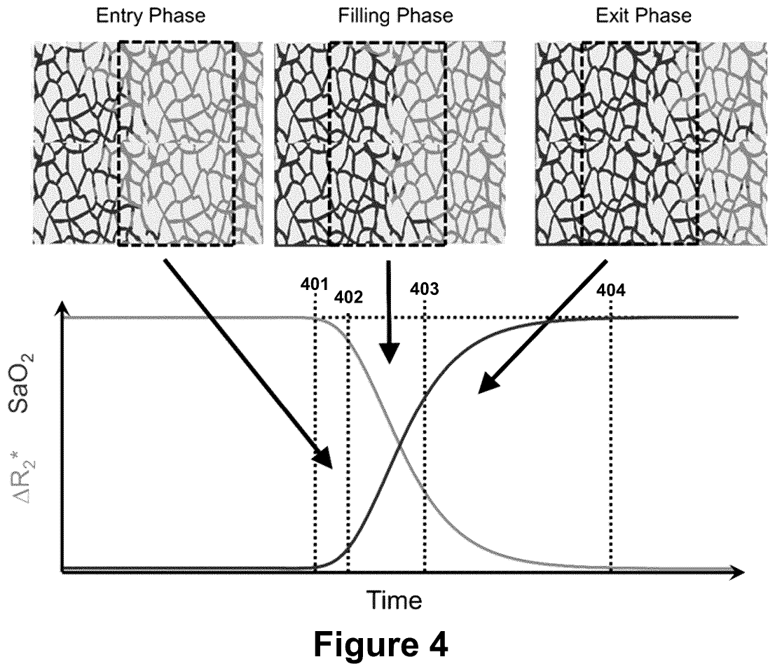

Instead, a novel model is proposed to explain the observed ΔR₂* signal response to a step increase in SaO₂. The contrast agent accumulation dynamics in a voxel can be viewed as the result of a distribution of contrast agent entry times in a population of capillaries, rather than the synchronous entry assumed in FIG. 4B. Even with the assumption of a step increase in hemoglobin oxygenation in all vessels, the arrival time of the saturation wavefront will vary among vessels entering the voxel as there is a range of blood flows and path distances to the voxel. This model is illustrated in FIG. 4. Once all vessels entering the voxel contain oxygenated blood, the ΔR₂* signal will decline linearly until the saturation wavefront of oxygenated blood in some vessels begins to leave the voxel. At that stage there is an exponentially decreasing rate of decline in the ΔR₂* signal, reaching an asymptote at a new steady signal value.

FIG. 4 is a graph illustrating the proposed model. The capillary diagrams show the phases of signal change as the wavefront of the step increase in SaO₂ in a population of vessels passes through a voxel to fill all vessels in the voxel with blood containing increased SaO₂. The graph shows the net increase in SaO₂ within the voxel (black line) and the resulting ΔR₂* signal (gray line) as the step change in SaO₂ in the population of capillaries reaches the voxel. At dotted line 401 in the graph, the entry phase illustrates the arrival of oxygenated blood (black line) displacing the hypoxic blood (gray line). Entry is complete in all capillaries at dotted line 402 in the graph and the filling phase is a constant rate of filling with oxygenated blood in all vessels, resulting from a net voxel flow of CBF and a linear decrease in ΔR₂* signal. At dotted line 403 in the graph, the linear stage of filling ends and the exit phase is characterized by the slowing of the net rate of increase in SaO₂ with ΔR₂* signal following a declining exponential pattern reaching an asymptote of zero change at dotted line 404.

According to the method provided herein, perfusion metrics can be computed directly from measurements of the ΔR₂* signal response to the step increase in SaO₂, assuming the descriptive model shown in FIG. 4 (the “THx-dOHb-Step analysis”). As demonstrated herein, this direct examination of the ΔR₂* signal step response enables the calculation of relative perfusion metrics without recourse to conventional deconvolution analysis and selection of an AIF and kinetic model.

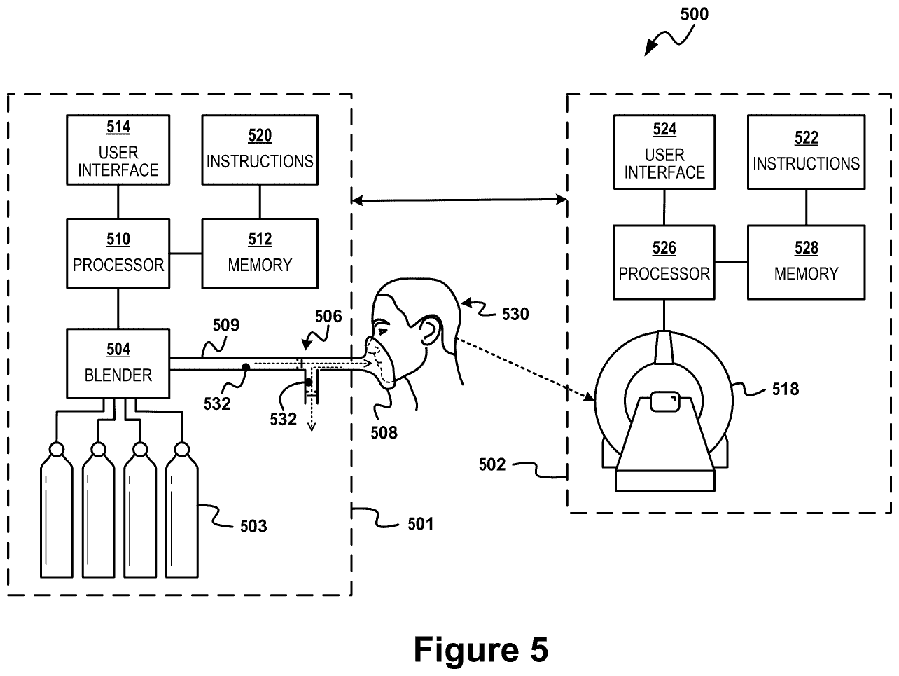

FIG. 5 shows a system 500 for quantifying a perfusion metric using deoxyhemoglobin as a contrast agent. The system 500 includes a respiratory device. Generally, the respiratory device comprises a means of delivering a hypoxic gas to a subject and subsequently delivering an oxygenated gas to the subject. In one example, the respiratory gas comprises an inspiratory limb with a three-way valve for delivering gas to the subject and an expiratory limb for receiving exhaled gases. The inspiratory limb is configured to provide a hypoxic gas to the subject comprising 10% oxygen. The balance of the hypoxic gas may comprise nitrogen. After inducing hypoxic in the subject, three-way valve is actuated to provide only oxygen or an oxygen-enriched gas to the subject, which generates higher hemoglobin saturation. In the examples described herein, the respiratory device is a sequential gas delivery (SGD) device 501 configured to provide delivery gases to a subject 530 and target an arterial partial pressure of a gas such as CO₂ or 02. Using the SGD device 501, targeted PₐO₂ values may be attained while maintaining normocapnia. The system 500 further includes a magnetic resonance imaging (MRI) system 502. The SGD device 501 includes gas supplies 503, a gas blender 504, a mask 508, a processor 510, memory 512, and a user interface 514. The SGD device 501 may be configured to control end-tidal partial pressure of CO₂ (P_{ET}CO₂) and end-tidal partial pressure of 02 (P_{ET}O₂) by generating predictions of gas flows to actuate target end-tidal values. The SGD device 501 may be an RespirAct™ device (Thornhill Medical™: Toronto, Canada) specifically configured to implement the techniques discussed herein. For further information regarding sequential gas delivery, U.S. Pat. No. 8,844,528, US Publication No. 2018/0043117, and U.S. Pat. No. 10,850,052, which are incorporated herein by reference, may be consulted.

The gas supplies 503 may provide carbon dioxide, oxygen, nitrogen, and air, for example, at controllable rates, as defined by the processor 510. A non-limiting example of the gas mixtures provided in the gas supplies 503 is:
a. Gas A: 10% O₂, 90% N₂;
b. Gas B: 10% O₂, 90% CO₂;
c. Gas C: 100% O₂; and
d. Calibration gas: 10% 02, 9% CO₂, 81% N₂.

The gas blender 504 is connected to the gas supplies 503, receives gases from the gas supplies 503, and blends received gases as controlled by the processor 510 to obtain a gas mixture, such as a first gas (G1) and a second gas (G2) for sequential gas delivery.

The second gas (G2) is a neutral gas in the sense that it has about the same composition as the gas exhaled by the subject 530, which includes about 4% to 5% carbon dioxide. In some examples, the second gas (G2) may include gas actually exhaled by the subject 530. The first gas (G1) has a composition of oxygen that is equal to the target P_{ET}O₂ and preferably no significant amount of carbon dioxide. For example, the first gas (G1) may be air (which typically has about 0.04% carbon dioxide), may consist of 21% oxygen and 79% nitrogen, or may be a gas of similar composition, preferably without any appreciable CO₂.

The processor 510 may control the gas blender 504, such as by electronic valves, to deliver the gas mixture in a controlled manner. The processor 510 may be configured to compute the compositions of the first gas (G1) and the second gas (G2) required to attain the target P_{ET}O₂ and the target P_{ET}CO₂. The processor 410 may compute the compositions of the first gas (G1) and the second gas (G2) according to a prospective targeting algorithm. The processor 410 may further compute the compositions of the first gas (G1) and the second gas (G2) according to feedback received from one or more sensors 532. In particular, the sensors 532 may measure the composition of an exhaled gas.

The mask 508 is connected to the gas blender 504 and delivers gas to the subject 530. The mask 508 may be sealed to the subject's face to ensure that the subject 530 only inhales gas provided by the gas blender 504 to the mask 508. In some examples, the mask is sealed to the subject's face with skin tape such as Tegaderm™ (3M™: Saint Paul, Minnesota). A valve arrangement 506 may be provided to the SGD device 501 to limit the subject's inhalation to gas provided by the gas blender 504 and limit exhalation to the room. In the example shown, the valve arrangement 106 includes an inspiratory one-way valve from the gas blender 504 to the mask 508, a branch between the inspiratory one-way valve and the mask 508, and an expiratory one-way valve at the branch. Hence, the subject 530 inhales gas from the gas blender 504 and exhales gas to the room.

The subject 530 may breathe spontaneously or be mechanically ventilated.

The gas supplies 503, gas blender 504, and mask 508 may be physically connectable by a conduit 509, such as tubing, to convey gas. Any suitable number of sensors 532 may be positioned at the gas blender 104, mask 408, and/or conduits 409 to sense gas flow rate, pressure, temperature, and/or similar properties and provide this information to the processor 510. Gas properties may be sensed at any suitable location, so as to measure the properties of gas inhaled and/or exhaled by the subject 530.

The processor 510 may include a central processing unit (CPU), a microcontroller, a microprocessor, a processing core, a field-programmable gate array (FPGA), an application-specific integrated circuit (ASIC), or a similar device capable of executing instructions. The processor 510 may be connected to and cooperate with the memory 512 that stores instructions and data.

The memory 512 includes a non-transitory machine-readable medium, such as an electronic, magnetic, optical, or other physical storage device that encodes the instructions. The medium may include, for example, random access memory (RAM), read-only memory (ROM), electrically erasable programmable read-only memory (EEPROM), flash memory, a storage drive, an optical device, or similar.

The user interface 514 may include a display device, touchscreen, keyboard, speaker, microphone, indicator, buttons, the like, or a combination thereof to allow for operator input and/or output.

Instructions 520 may be provided to carry out the functionality and methods described herein. The instructions 520 may be directly executed, such as a binary file, and/or may include interpretable code, bytecode, source code, or similar instructions that may undergo additional processing to be executed. The instructions 520 may be stored in the memory 512.

The system 500 further includes an MRI system 502 for conducting magnetic resonance imaging on the subject 530. A suitable MRI device may include a scanner 518 such as a 3-tesla (3T) MRI scanner or a 7-tesla (7T) MRI scanner. A suitable example of a 3T MRI scanner is the Signa HD×t 3.0T™, provided by GE Healthcare (Milwaukee, USA). A suitable example of 7-tesla MRI scanner is the MAGNETOM™ 7T MRI, provided by Siemens (Munich, Germany). In addition to the scanner 418, the MRI system 402 may further include a processor 526, a memory 528, and a user interface 524.

Any description of the processor 526 may apply to the processor 510 and vice versa. Likewise, any description of the memory 528 may apply to the memory 512 and vice versa. Similarly, any description of the instructions 522 may apply to the instructions 520 and vice versa. Also, any description of user interface 524 may apply to user interface 514, and vice versa. In some implementations, the MRI system 502 and the SGD device 501 share one or more of a memory, processer, user interface, and instructions, however, in the present disclosure, the MRI system 502 and the SGD device 501 will be described as having respective processors, user interfaces, memories, and instructions. The processor 410 of the SGD device 501 may transmit data and instructions to the processor 526 of the MRI system 502. The processor 526 of the MRI system 502 may transmit data and instructions to the processor 510 of the SGD device 501. The system 500 may be configured to synchronize MRI imaging obtained by the MRI system 502 with measurements obtained by the SGD device 501.

The processor 526 may retrieve operating instructions 522 from the memory 528 or from the user interface 524. The operating instructions 522 may include image acquisition parameters. The parameters may include a pre-determined number of contiguous slices, a defined isotropic resolution, a diameter for the field of view, a repetition time (TR), and an echo time. Various protocols may be employed such as multi-echo T2* (ME-T₂) imaging. According to a non-limiting example of multi-echo T2* parameters, the voxel resolution is 3 mm×3 mm×3 mm, the repetition time (TR) is 1500 ms, the first echo time (TE1) is 10.7 ms, the second echo time (TE2) is 272 ms, and the third echo time (TE3) 43.6 ms.

The user interface 524 may include a display device, touchscreen, keyboard, speaker, indicator, microphone, buttons, the like, or a combination thereof to allow for operator input and/or output. Data generated and images acquired by the processor 526 may be displayed at the user interface 524.

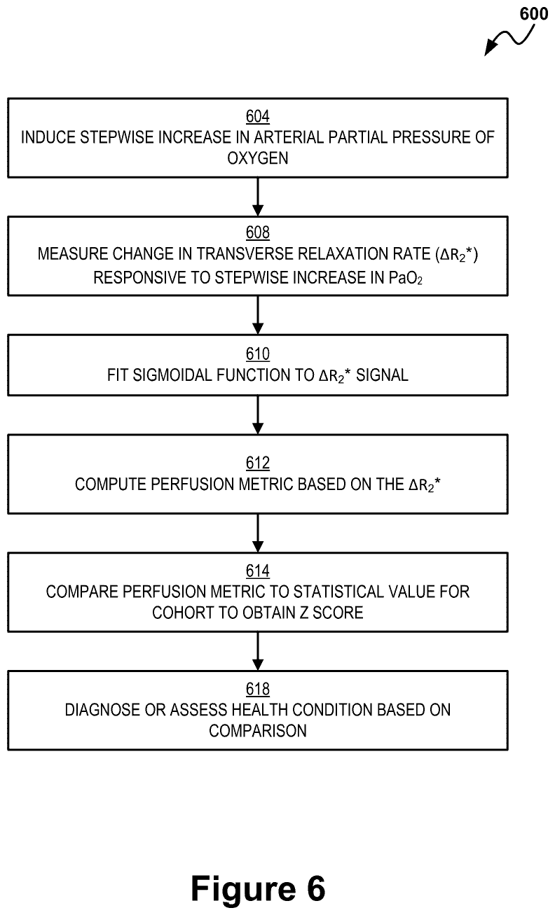

FIG. 6 shows an example method 600 of directly computing a perfusion metric using a stepwise change in deoxyhemoglobin. The method 600 may be performed using the system 500, however the method 600 is not particularly limited.

Block 604 comprises inducing a stepwise increase in arterial partial pressure of oxygen (PₐO₂) in the subject. Block 604 is performed by the respiratory device which delivers a hypoxic gas to induce hypoxia in the subject's arterial blood and then delivers an oxygenated gas to reoxygenate the subject's arterial blood.

To attain hypoxia, the respiratory device may deliver successive tidal volumes of hypoxic gas to the subject 530 over a series of breaths. The successive volumes of hypoxic gas gradually dilute the oxygen in the subject's functional residual capacity (FRC) until the subject's arterial blood becomes hypoxic. To reoxygenate the arterial blood, the respiratory device may deliver an oxygenated gas to reoxygenate the arterial blood within a single breath. Thus, the reoxygenation step is an abrupt change in PₐO₂.

In examples where the respiratory device is an SGD device, block 604 is performed by the SGD device 501 which controls the end-tidal partial pressure of oxygen (P_{ET}O₂) using a prospective targeting algorithm. The SGD device 501 may also control the end-tidal partial pressure of carbon dioxide (P_{ET}CO₂) using the prospective targeting algorithm. Using the prospective targeting algorithm, the SGD device 501 targets a first P_{ET}O₂ to induce hypoxia and then targets a second P_{ET}O₂ which is higher than the first P_{ET}O₂ in order to reoxygenate the subject's arterial blood, causing the stepwise increase in PₐO₂.

The difference between the first and second P_{ET}O₂ may be sufficient to induce a measurable decrease in the concentration of deoxyhemoglobin. In some examples, the first P_{ET}O₂ induces hypoxia in the subject while the second P_{ET}O₂ induces normoxia, however the method 600 is not particularly limited. In other examples, both the first and second P_{ET}O₂ are selected to induce varying levels of hypoxia. The quality of the signal may be affected if the second P_{ET}O₂ induces hyperoxia, since hyperoxia can cause oxygen to dissolve in blood.

In some examples, the first P_{ET}O₂ is below 80 mmHg, below 70 mmHg, below 60 mmHg, or below 50 mmHg. In some cases, the first P_{ET}O_{2 2} is between 30 mmHg and 70 mmHg. In yet further examples, the first P_{ET}O₂ is approximately 50 mmHg, approximately 45 mmHg, approximately 40 mmHg, approximately 35 mmHg, or approximately 30 mmHg. In some examples, the second P_{ET}O₂ is greater than 70 mmHg. In further examples, the second P_{ET}O₂ is greater than 80 mmHg, greater than 90 mmHg, or greater than 100 mmHg, greater than 105 mmHg, or greater than 110 mmHg. In some cases, the second P_{ET}O₂ is between 70 mmHg and 110 mmHg. In additional examples, the second P_{ET}O₂ is approximately 70 mmHg, approximately 75 mmHg, approximately 80 mmHg, approximately 85 mmHg, approximately 90 mmHg, approximately 95 mmHg, or approximately 100 mmHg. In a specific, non-limiting example, the first P_{ET}O₂ is about 40 mmHg and the second P_{ET}O₂ is about 95 mmHg.

As a further part of block 604, the SGD device 501 may maintain the targeted P_{ET}O₂ values for respective periods of time. In a particular, non-limiting example the SGD device 401 maintains the first P_{ET}O₂ for about 60 seconds and maintains the second P_{ET}O₂ for about 60 seconds. In other examples, the SGD device 401 maintains the first P_{ET}O₂ for about 30 seconds and maintains the second P_{ET}O₂ for about 30 seconds. In some examples, the SGD device 501 maintains the first P_{ET}O₂ for less than 15 seconds and maintains the second P_{ET}O₂ for less than 15 seconds. Generally, the hypoxia should not be maintained for long before reoxygenation, to avoid confounding increases in CBF. The CBF response may be slow, but there is high variability between individuals.

To obtain multiple measurements at block 608, block 604 may be repeated to induce a series of stepwise changes in PₐO₂.

Due to gas exchange in the alveoli, the P_{ET}O₂ and P_{ET}CO₂ are approximately the same as the PₐO₂ and PaCO₂, respectively. Reoxygenating the subject's blood from hypoxia to normoxia may be achieved within one breath, and in some examples less than one second, using the SGD device 501. The change in signal as oxygenated blood displaces the deoxygenated blood in the voxel reflects the hemodynamic parameters in a region of the subject's body, which can be measured.

Block 608 comprises measuring a magnetic signal in a selected voxel and deriving a change in effective transverse relaxation rate (ΔR₂*) responsive to the step-wise increase in PₐO₂ imposed at block 604. In system 500, block 608 is performed by the MRI system 502 which measures a magnetic signal in the subject 530 while the respiratory device is controlling the subject's PₐO₂.

As part of block 608, the MRI system 502 measures one or more T2*-weighted signals in the subject and computes the ΔR₂* based on the T2*-weighted signal. For exemplary purposes, the method 600 may be explained herein with respect to one T2*-weighted signal which may be measured in a selected voxel, however it should be understood that the MRI system 502 generally measures a plurality of T2*-weighted signals in a plurality of voxels, including the selected voxel. The MRI system 502 may measure the T2*-weighted signals by performing a T2*-weighted scan of the subject 530. The parameters of the T2*-weighted scan may include TR=1500 ms, TE=30 ms, flip angle=73°, 29 slices, voxel size=3 mm isotropic with 64×64 matrix, however the parameters of the T2*-weighted scan are not particularly limited and other parameters may be suitable.

As a further part of block 608, the processor 526 may preprocess the T2*-weighted signals. Preprocessing may include volume registering the T2*-weighted signals. Preprocessing may further include slice-time correcting the T2*-weighted signals. Preprocessing may further include co-registering the T2*-weighted signals to anatomical images. Preprocessing may further include removing noise from the T2*-weighted signals. Preprocessing may further include applying a spatial blur to the T2*-weighted signals. In particular examples, the processor 426 applies AFNI software to co-register the T2*-weighted signals to anatomical images (National Institutes of Health, Bethesda, Maryland, Version AFNI_24.0.12 ‘Caracalla’ URL https://afni.nimh.nih.gov).

As a further part of block 608, the processor 526 derives the ΔR₂* based on the T2*-weighted signal. The T2*-weighted signal may be computed into ΔR₂* using Equation 3:

$$Δ {R}_{2}^{*} ( t ) = ( \frac{- 1}{TE} ) \times ln \{ \frac{S ( t )}{S ( 0 )} \} \qquad ( 3 )$$

Since the MRI system 502 measures the magnetic signal while the respiratory device is inducing the stepwise change, block 608 produces a time course of ΔR₂* values for the selected voxel.

Block 610 comprises fitting a predetermined sigmoid function to the ΔR₂* values derived at block 608. In system 500, block 610 is performed by the processor 526 which retrieves the predetermined sigmoid function from memory and optimizes parameters of the sigmoid function to reduce error between the function and the ΔR₂* values.

The predetermined sigmoid function may include one or more parameters defining its amplitude, inflection point, slope, and offset. The optimization may be performed using a curve fitting algorithm, such as least squares minimization.

In some examples, the predetermined sigmoid function is symmetrical. In particular examples, the predetermined sigmoid function is a Gompertz fit function. The Gompertz fit function may be defined using Equation 4:

$${S}_{fit} ( t ) = {S}_{base} + a {e}^{- {be}^{- ct}} \qquad ( 4 )$$

$$where :$$

$$S = Δ {R}_{2}^{*}$$

$$t = time$$

$${S}_{fit} ( t ) = the fitted Δ {R}_{2}^{*} signal time course of the step response$$

$$exp = power of e$$

$${S}_{base} = the initial value of {S}_{fit} ( t )$$

$$a = the magnitude of the S decrease$$

$$b = the displacement along the time axis$$

$$c = the rate of change$$

In some examples, the predetermined sigmoid function is fitted to a portion of the ΔR₂* values derived at block 608. As part of block 610, the processor 526 may select the ΔR₂* values that coincide with the stepwise increase in PₐO₂. The portion of the ΔR₂* values may be selected based on user inputs received at the user interface 524.

Block 612 comprises computing a perfusion metric based on the ΔR₂* measured at block 608. In system 500, block 608 is performed by the processor 526. In examples where the method 600 includes block 610, the perfusion metric may be computed based on the sigmoid function. The perfusion metric may include one or more of rCBV, rCBF, MTT, and rBAT, however the perfusion metric is not particularly limited.

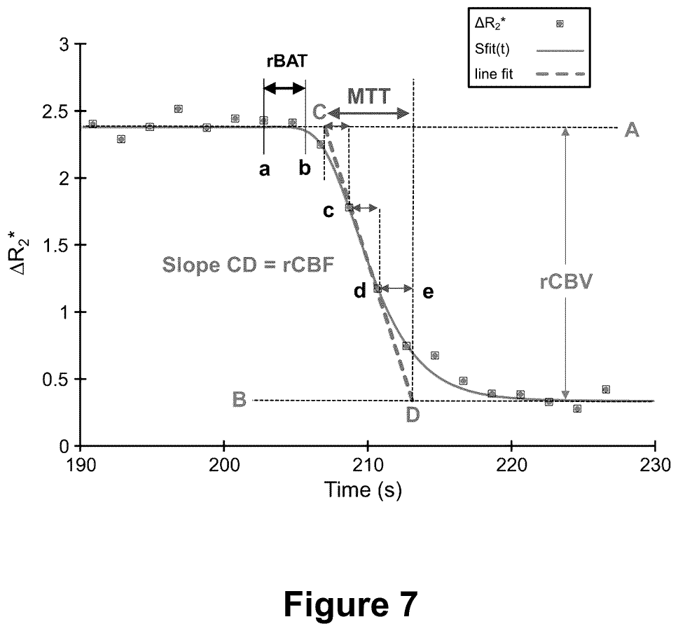

FIG. 7 is a graph illustrating exemplary performance of blocks 608, 610, and 612. In FIG. 7, the ΔR₂* is plotted against time. The solid line shows the sigmoid function, which in this example is a Gompertz fit function fitted to the ΔR₂*. The amplitude of the Gompertz fit function is defined by line A and line B. Line CD is a tangent line at the inflection point of the sigmoid function, and the slope of line CD is the maximum rate of decrease in the sigmoid function. The mean transit time (MTT) can be calculated as the time range of the tangent line. The relative cerebral blood volume (rCBV) can be calculated as the amplitude of the sigmoid function. The relative cerebral blood flow (rCBF) can be calculated as the slope of the tangent line or the maximum rate of decrease in the sigmoid function. Reference time (a) corresponds to a time before the ΔR₂* begins to decrease in response to the stepwise increase in PₐO₂. Start time (b) indicates where the ΔR₂* has decreased by 2% of the rCBV. The relative blood arrival time (rBAT) can be calculated as the difference between the start time (b) and the reference time (a), with negative values signifying earlier arrival.

The maximum rate of decrease of the ΔR₂* may be calculated from the Sfit(t) parameters as “a×c/e” to measure rCBF, where e is the base of natural logarithms. A tangent line with this slope is drawn through the time of maximum slope, “In (b)/c” (FIG. 7 at CD). The tangent line defines three temporal regions, as indicated by the arrows in FIG. 7. First, the exponential increase in the rate of decline of the ΔR₂* as the step change in SaO₂ arrives at the voxel until the change has entered the voxel in all capillaries; second, a linear portion of the ΔR₂* decline as all vessels fill with the change in SaO₂ until the change begins to leave the voxel; third, an exponential decay in the rate of decline of the ΔR₂* as the SaO₂ change leaves the voxel. MTT is the sum of the time constants of the first and third temporal regions plus the time taken in the second linear ΔR₂* decrease temporal region. Consequently, MTT satisfies the central volume theorem as the ratio of CBV/CBF. Values of rCBV and rCBF were respectively multiplied by 2 and 200 to obtain easily readable values within the range of absolutes measures.

As part of block 612, the processor 526 may generate one or more perfusion maps comprising the calculated perfusion metrics for a plurality of voxels. In particular embodiments, the processor 526 transforms the perfusion map into Montreal Neurological Institute (MNI) space and overlays the perfusion map onto their respective anatomical images.

In examples where blocks 604 and 608 are repeated to obtain multiple ΔR₂* values, block 612 may further comprise computing a statistical value for the perfusion metric. In exemplary embodiments, the ΔR₂* is repeatedly obtained for a plurality of voxels to produce one or more perfusion maps which comprise statistical values for the respective perfusion metric.

Block 614 comprises comparing a perfusion metric to a statistical value for a reference population. In system 500, block 614 is performed by the processor 526 which compares perfusion metrics for the subject and the reference population.

In some examples, the reference population comprises a healthy group of subjects selected exhibiting no chronic illness or disease. In further examples, the reference population comprises a group of subjects exhibiting a health condition or disease. In yet further examples, the reference population comprises a group of subjects receiving a treatment. In some examples, the comparison at block 614 may be repeated by comparing the subject to two or more reference populations, for example a diseased population and a healthy population. It should be understood that the statistical value for the reference population is generated by performing blocks 604 to 612 on the group of subjects in the reference population and then combining the perfusion metrics generated for the reference population to obtain the statistical value. In non-limiting examples, the statistical value is an average of the perfusion metrics generated for the reference population. It should be further understood that the comparison at block 614 is most effective if the same or similar parameters are employed to generate the statistical value, for instance, the perfusion metric for the subject and the statistical value for the reference population should be obtained from measurements on corresponding voxels.

As part of block 514, the processor 526 may calculate a z-score representing the comparison between the perfusion metric for the subject and the statistical value for the reference population. The z-score for a plurality of voxels can be mapped to an anatomical image to obtain a z-score map.

In some examples, method 500 further includes drawing an interference based on the comparison between the subject and the reference population. Block 618 comprises assessing health condition or treatment based on the comparison at block 614. In system 500, block 618 is performed by the processor 526 which assesses a health condition or treatment based on the subject's perfusion metrics.

The health condition may include a cardiovascular disease or neurological disease selected from: Parkinson's disease, stroke, hemangiomas, vascular tumor or cyst, coronary heart disease, Moyamoya disease, Cerebral Venous Thrombosis, Arteriovenous Malformation, arterio-venous fistulas, angioma formation, carotid artery disease, intracranial hypertension, steno-occlusive disease, and kidney insufficiency, however the health condition is not particularly limited. In some alternatives, block 618 comprises diagnosing the health condition based on the z-score.

The treatment may include vasodilators, vasoconstrictors, anti-angiogenic agents, thrombolytics, chemotherapeutic, surgical procedures, intermittent hypoxia, exercise, diet, hydration, radiation therapy, brain stimulation, and neuromodulation, however the treatment is not particularly limited.

As part of block 618, the diagnosis or assessment may be output at the user interface 424. In some examples, block 618 is omitted from method 600.

In some examples, block 618 includes tissue or vessel classification. Based on the perfusion metric, the processor 526 may identify the tissue present in the selected voxel or classify the voxel as venous or arterial.

In view of the above, it will now be apparent that variants, combinations, and subsets of the foregoing embodiments are contemplated. For example, while the method 500 has been described with respect to a sequential gas delivery device, other respiratory devices may be used to deliver hypoxic gas and reoxygenate the blood in a step-wise fashion. Furthermore, the steps of the method 600 may be performed in other orders, and some steps may be omitted. Additionally, the method 600 and system 500 have been described in respect to measurements of the brain, but the method and system may be similarly applied to other organs or regions of the body such as kidney, heart, skeletal muscle, and the like.

It will now be apparent to a person of skill in the art that the present specification affords certain advantages over the prior art. In particular, perfusion metrics derived directly from ΔR₂* offer advantages over those calculated using an arterial input function (AIF) by reducing computational burden and improving processing efficiency within the computing environment. Unlike AIF-based approaches, which require extraction of a representative vascular signal and execution of deconvolution algorithms involving regularization and parameter tuning, the disclosed method computes perfusion metrics directly from a time-dependent ΔR₂* signal using a predetermined sigmoid function. This avoids numerically unstable inverse operations and eliminates the need for high-volume voxel-wise deconvolution. As a result, the method reduces processor load, minimizes memory usage, and facilitates faster and more scalable implementation on clinical or embedded computing platforms. In further contrast to AIF-based methods which require an operator to identify and select a reference voxel in the middle cerebral artery, the present method is faster and improves inter-scan repeatability.

### EXAMPLES

Perfusion metrics obtained with the method 500 are compared to those generated from GBCA-AIF and THx-dOHb-AIF analyses using the instantaneous homogeneity kinetic model (FIG. 3A), in seven healthy study subjects. This comparison is necessarily qualitative, as the comparator proxy measures are not considered gold standard hemodynamic measures.

### 1 Results

### 1.1 Revascularization Signal Noise

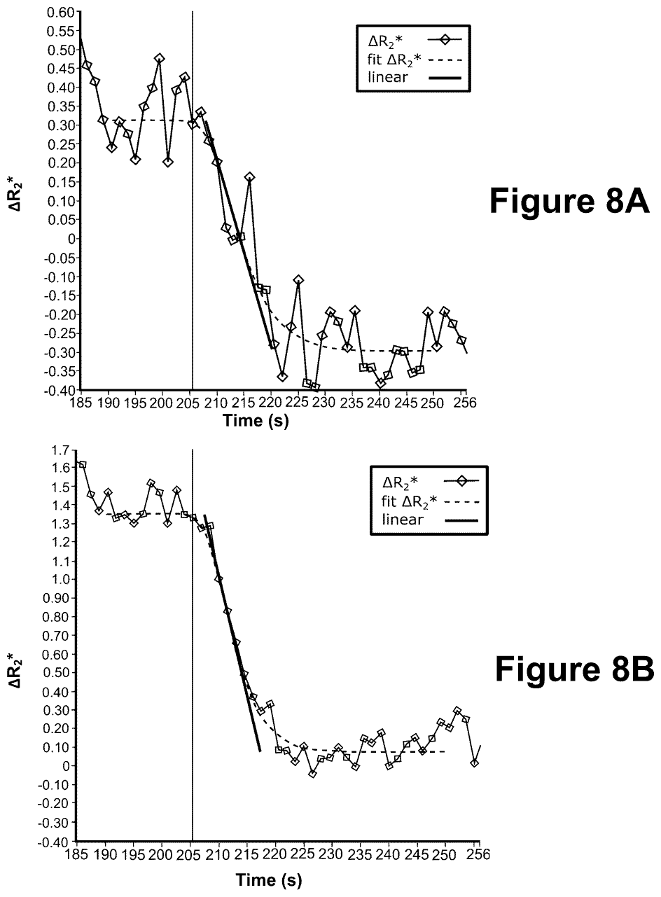

FIGS. 8A and 8B illustrate the variation in the ΔR₂* signal noise among voxels and shows how the fitting of a Gompertz function is used to provide a best fit to the data. The images are graphs obtained from the step analysis computer program. FIG. 8A is representative of a noisy signal, with FIG. 8B showing a less noisy signal. The dotted line is the fit of the Gompertz function to the ΔR₂* signal response to a step change in SaO₂. The solid line is fitted to the linear portion of the function and extended to baseline (top) and asymptote of oxygenated blood (bottom). The vertical line is the reference time cursor used for all voxels to calculate blood arrival time. (The analysis method is described with respect to FIG. 7).

### 1.2 Group Comparisons

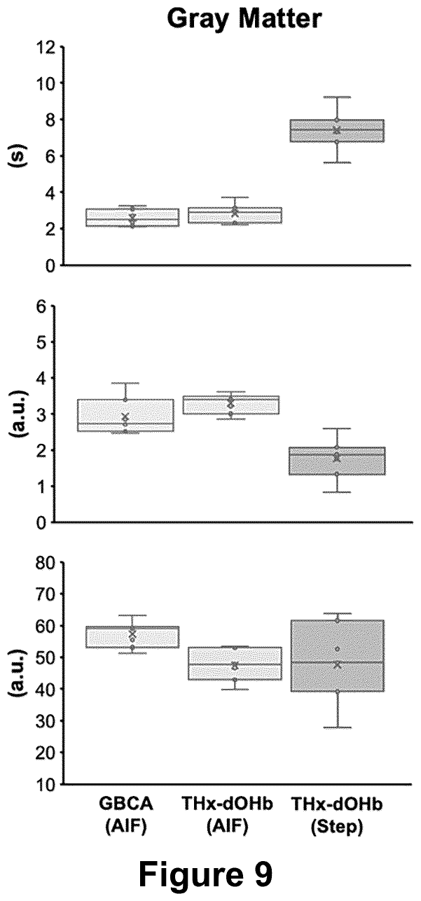

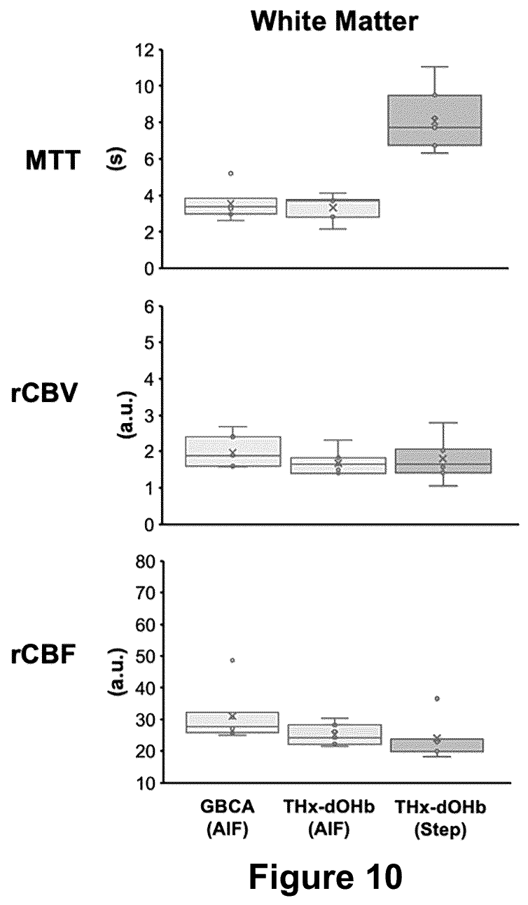

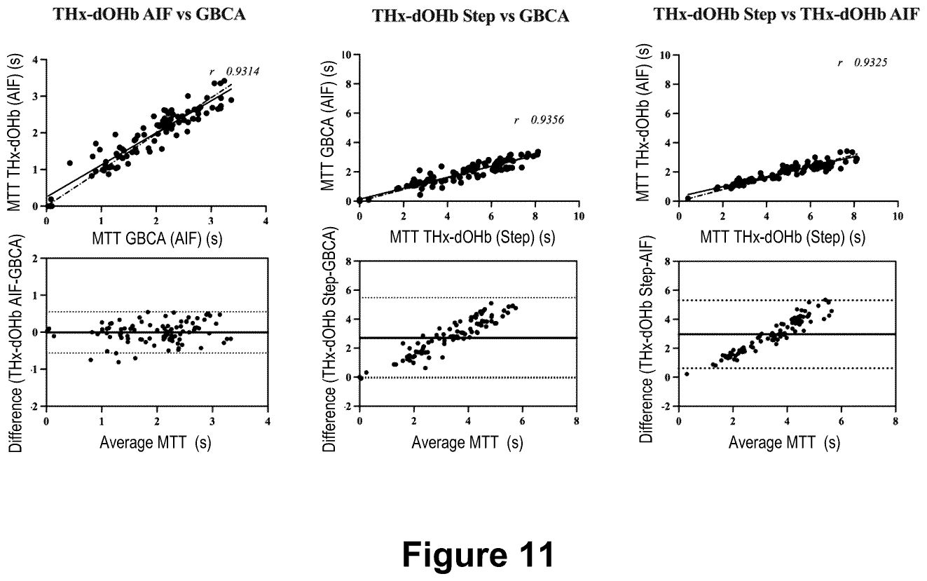

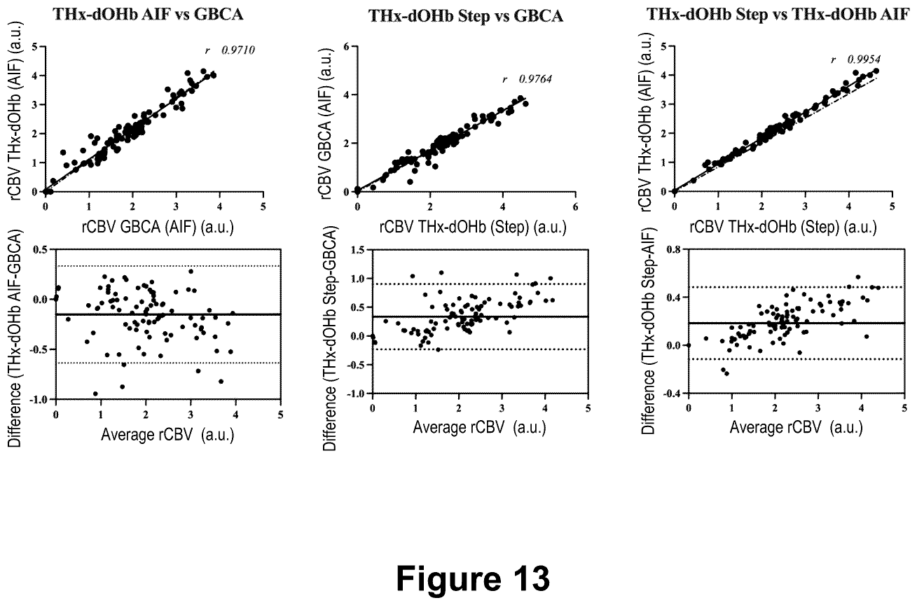

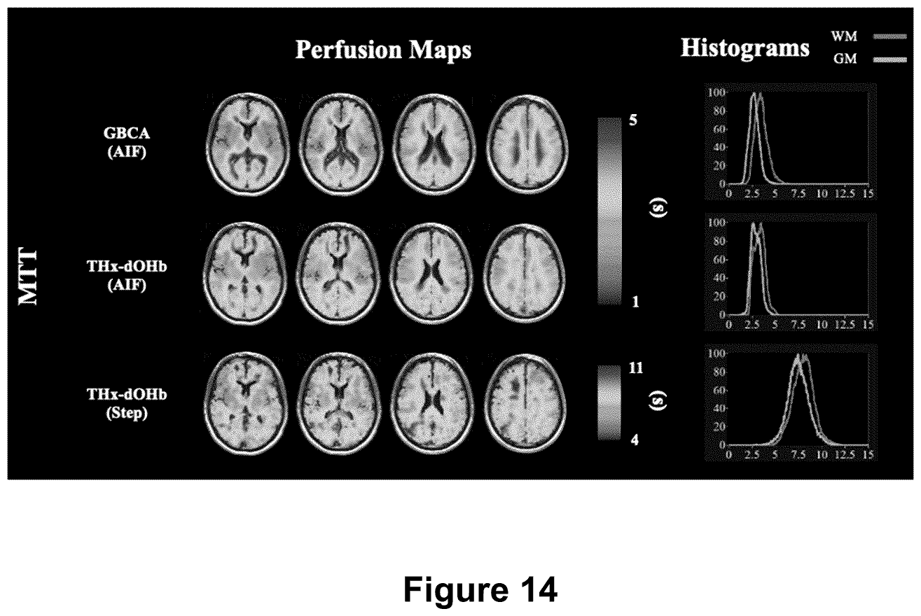

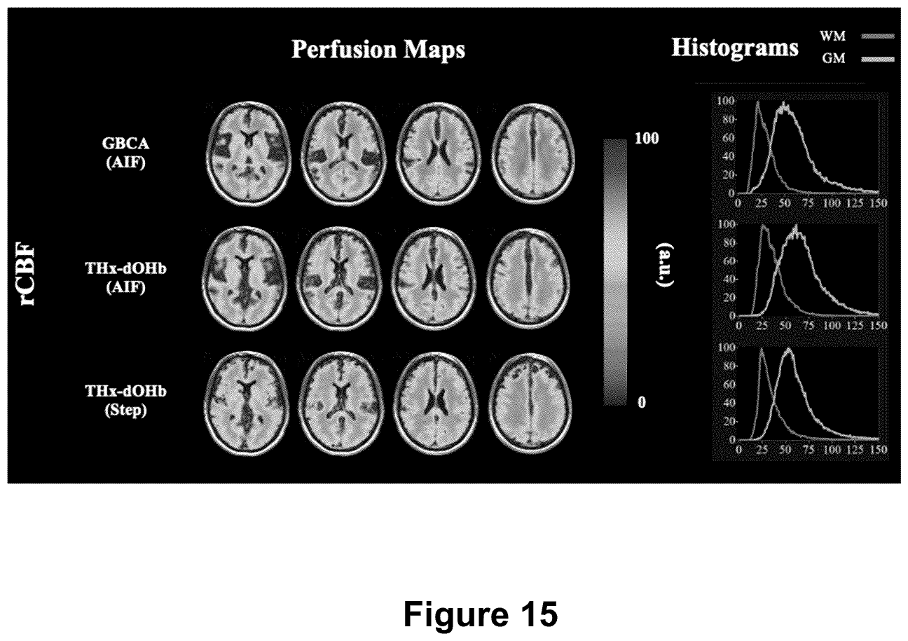

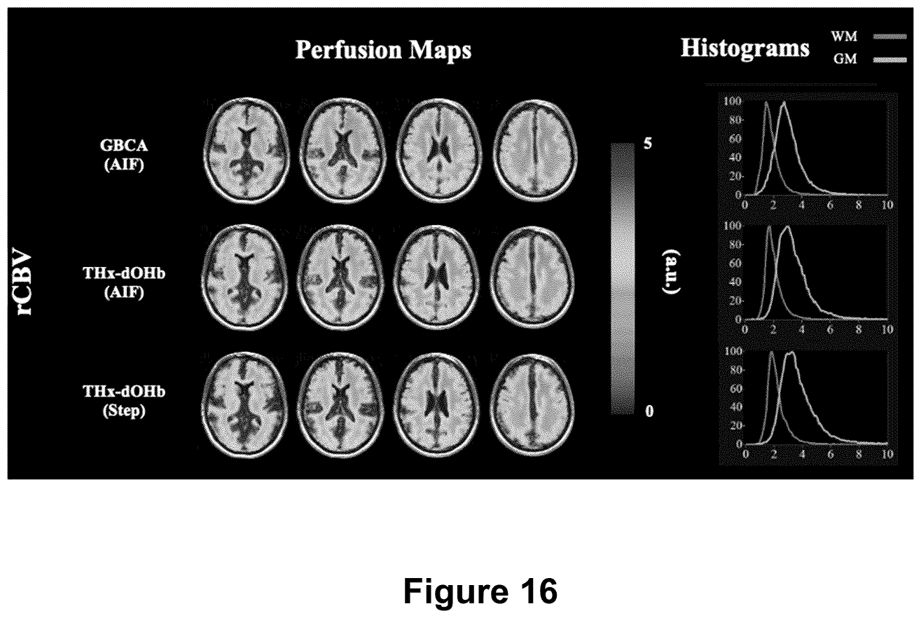

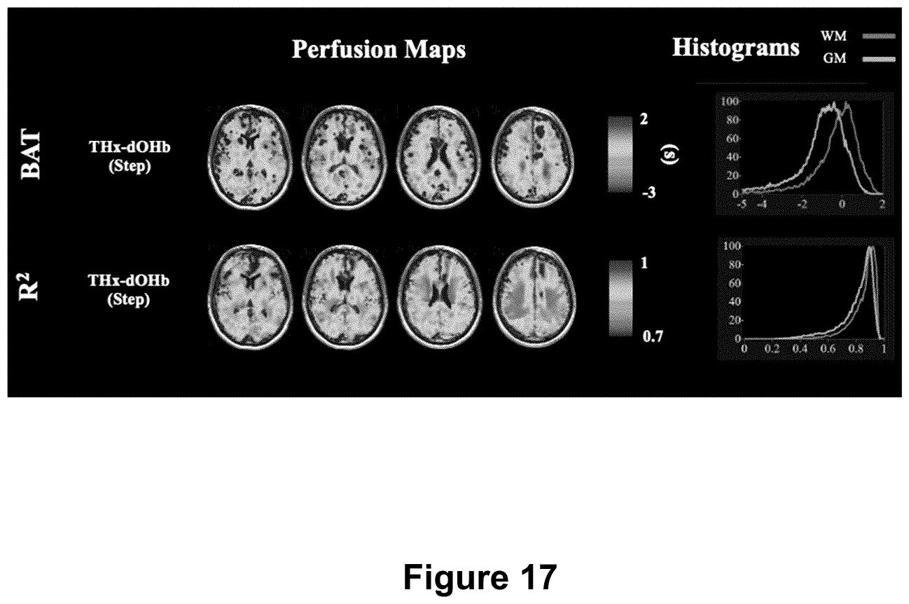

FIGS. 9 and 10 compares the group averaged metrics from the three analyses in the healthy subjects using boxplots. FIGS. 11 to 13 show scatter and Bland-Altman plots. FIGS. 14 to 17 display axial slices of the group averaged perfusion metrics and their associated GM and WM histograms.

FIGS. 9 and 10 show the distribution of the group perfusion metrics in the gray matter (FIG. 9) and white matter (FIG. 10) for the three analyses for the 7 healthy subject group. Note the prolonged MTT from the step analysis (see Discussion for an explanation). (GBCA, gadolinium-based contrast agent; THx-dOHb, transient hypoxia induced deoxyhemoglobin; AIF, arterial input function; rCBF, relative cerebral blood flow; rCBV, relative cerebral blood volume; MTT, mean transit time.)

FIGS. 11 to 13 show the regional agreement of grouped perfusion metrics between the three analyses. Correlation and Bland-Altman limits of agreement analyses using one hundred (100) regions of interest between pairs of perfusion measurements.

FIGS. 14 to 17 show representative axial slices. The healthy subject group metrics (rCBF, rCBV and MTT) obtained using the three analyses with their associated whole brain histograms for gray matter, GM (red) and white matter, WM (green). The THx-dOHb-Step analysis also provides an R² goodness of fit measure and a blood arrival time relative to the reference cursor time; negative values indicating arrival before the reference cursor time, positive after.

The grouped mean (SD) hemodynamic ratios for GM-to-WM are presented in Table 1. The GBCA-AIF rCBV GM-to-WM ratio was significantly lower than those for THx-dOHb-AIF and THx-dOHb-Step (P=0.013 and 0.018 respectively), while the GM-to-WM ratios for MTT and rCBF did not differ between analyses.

TABLE 1
Grouped mean (SD) GM-to-WM ratios.

| Metric | THx-dOHb-Step | THx-dOHb-AIF | GBCA-AIF | P |
|---|---|---|---|---|
| MTT | 0.95 (0.2) | 0.86 (0.11) | 0.75 (0.13) | 0.11 |
| rCBF | 2.22 (0.65) | 1.92 (0.32) | 1.93 (0.40) | 0.44 |
| rCBV | 1.96 (0.34) | 2.01 (0.30) | 1.51 (0.19) | 0.008 |

A two-way ANOVA of MTT values with factors GM-to-WM and type of analysis found that the MTT values for the THx-dOHb-Step analysis were greater in WM than GM for all three analyses (P=0.016). MTT values for the THx-dOHb-Step analysis were greater than those for the other two analyses (P<0.001), which did not differ (P=0.971).

### 2 Discussion

The main findings of this study are that the hemodynamic parameters obtained by the analysis of the voxel ΔR₂* changes following a step re-oxygenation produced similar distributions of relative hemodynamic measures obtained using GBCA and THX-dOHb contrasts analyzed using existing kinetic model-based deconvolution analysis involving an AIF. We believe the similarity of the patterns of the hemodynamic measures implies widespread correspondence throughout the brain of the relative blood flow and volume. Nevertheless, the strongest arguments supporting the hypothesis of validity of the THx-dOHb-Step method are the full visual and mathematical traceability of the process from signal detection to hemodynamic calculation, and the physiological authenticity of the vascular model it follows (FIG. 4).

2.1 Group Metrics for rCBF and rCBV

The resting perfusion group metrics for rCBF and rCBV for all three analyses are relative measures. Consequently, comparisons to published absolute values are not possible, leaving only comparisons of similarity of distribution to relative values measured by deconvolution and AIF. The group averaged maps of rCBV and rCBF in FIGS. 14 to 17 show similar patterns of distribution of their measured parameters, which are corroborated in the scatter and Bland-Altman plots in FIGS. 9 and 10. Of particular significance is that they are also strikingly similar in regional distribution to published group average maps of these metrics generated using other analytical methods.

The GM-to-WM ratios for rCBF and rCBV were compared between analysis techniques and found that the GM-to-WM ratio for rCBF did not differ between analyses techniques. GM to WM ratios for rCBF reported in the literature range from 2:1 to 3:1 in healthy subjects. One study found the GM-to-WM ratio to be 3.34±0.48 to 1 when using CT perfusion, and about 2.78±0.25 to 1 when using PET. This finding indicates that rCBF is significantly higher in gray matter compared to white matter, and is consistent with the findings of this current study. The GM-to-WM ratios for GBCA-AIF rCBV were significantly lower than those for THx-dOHb-AIF and THx-dOHb-Step analyses (P=0.013 and 0.018 respectively). The GM-to-WM ratio for rCBV calculated using THx-dOHb match literature values of about 2 to 1 in healthy subjects. However, the rCBV GM-to-WM ratio calculated using GBCA is slightly lower (ratio of 1.5 to 1) in comparison to published literature values (˜2 to 1). A study by Wenzel et al. found the GM-to-WM ratio to be ˜2.1±0.5 to 1 and Artzi et al. found the GM-to-WM ratio to be ˜2.38 to 1 both using DSC-MRI.

### 2.2 Group Metrics for MTT

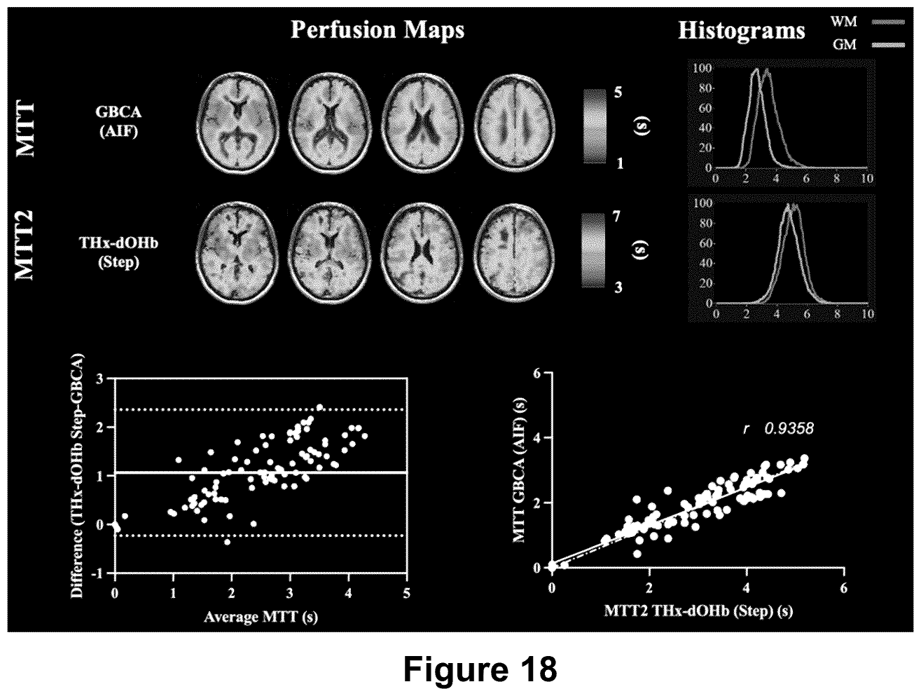

The MTT values for the THx-dOHb-Step analysis are systematically larger than those measured using an AIF and deconvolution. Note that in our model of the THx-dOHb-Step there is a variation in the time of entry of contrast into the voxel (C-D in FIG. 7). We include this ΔR₂*effect of the distribution of the times of entry of the step SaO₂ change in vessels arriving at the voxel in the calculation of MTT, which results in longer values. For both AIF-deconvolution analyses, the initial entry time of the SaO₂ step change is assumed to be instantaneous for all vessels passing through the voxel. Thus, the longer MTT values from the early arrived contrast is not accounted for (FIG. 3B). Indeed, excluding the time constant of the entry time from the THx-dOHb-Step calculation of MTT results in values falling within the range of the other analyses (FIG. 18) consistent with the shorter MTT values calculated for the GBCA-AIF analyses. The MTT GM-to-WM ratios were compared between analysis techniques. The GM-to-WM ratios varied slightly between analyses techniques, nevertheless, the MTT was always longer in WM than in GM (P=0.016). Previously published GM-to-WM MTT ratios measured by DSC-MRI suggest a ratio ranging between 0.5-0.8 to 1, similar to findings of this study, indicating that MTT is consistently longer in WM compared to GM.

FIG. 18 shows a comparison of group MTT maps and histograms comparing THx-dOHb-Step MTT2 to that of GBCA-AIF, with scatter and Bland-Altman plots. MTT2 is calculated from the THx-dOHb-Step response without the inclusion of the initial entry time constant. The color scales ranges were adjusted to obtain the highest contrast.

### 2.3 the Step Function

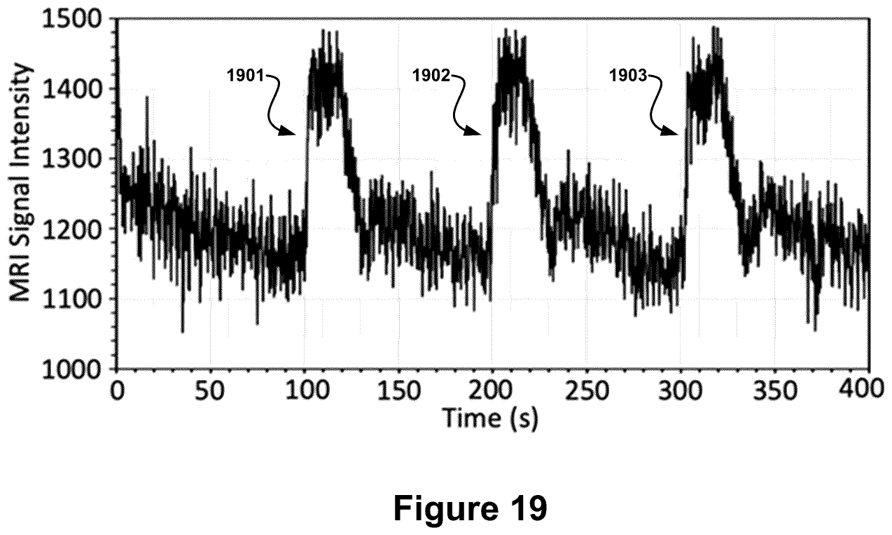

The use of rapid conversion of dOHb to oxyhemoglobin to generate a step function has been previously reported in mechanically ventilated rats in studying renal hemodynamics. Unfortunately, due to a large apparatus dead space, their tissue reoxygenation took 30 s compared to the 15-20 s in the present disclosure (FIG. 8 and FIG. 19). Furthermore, Zhao et al. employed oxygen changes from 10% O₂ (PO2 about 75 mmHg) to 100% (PO2 about 714 mmHg) for reoxygenation. This high PO2 is unnecessary for re-saturation of hemoglobin as it is almost fully saturated at a PO2 of about 90 mmHg. Finally, this high PO2 in the FRC of the rats after re-oxygenation resists deoxygenation by dilution of gas in the lungs, which is required for a repeat test. Here, our ability to precisely limit the PO2 in the functional residual capacity to full re-oxygenation without hyperoxia has provided this study the ability to rapidly generate repeat episodes of transient hypoxia and generate step increases in SaO₂ during re-saturation.

Maximizing accuracy and precision of hemodynamic measures by step re-oxygenation

### 2.4 GBCA Arterial Input Function.

The administration of GBCA and use of indicator dilution kinetics has been a mainstay of hemodynamic measures. The pharmacokinetic limitations of this approach follow from the uncontrolled dispersion of the contrast after injection and while flowing to and passing through the heart. No relationship between signal and contrast concentration can be discerned. Hemodynamic measurements must therefore be performed using AIF, deconvolution, and indicator dilution kinetics.

### 2.4.1 Deoxyhemoglobin Vs GBCA

Deoxyhemoglobin is paramagnetic and has also been used as a contrast agent. The advances over GBCA are as follows:
i) dOHb is generated noninvasively and then dissipated with re-oxygenation; therefore, repeatable within seconds. By reproducibly targeting [dOHb] in a subject via the RespirAct™, repeated studies are comparable and can facilitate longitudinal hemodynamic monitoring or the comparison of metrics between individuals.
ii) dOHb does not leak outside the vessels when measuring blood flow in disorders associated with disruption of the blood-brain-barrier, in contrast to GBCA where leakage correction methods must be applied. Therefore, dOHb should provide more accurate blood flow metrics in the setting of blood-brain-barrier disruption.
iii) [dOHb] in the blood and the resulting ΔR₂* signal can both be measured.

Nevertheless, until now the calculation of hemodynamic parameters has still required designation of an AIF and use of deconvolution calculations based on a hypothetical voxel perfusion model (FIG. 3A and FIG. 3B). Consequently, in this study as in previous studies, GBCA-AIF and THx-dOHb-AIF produce similar hemodynamic measures (FIGS. 9 to 17). We suspect that the similarity of the calculated hemodynamic measures from THx-dOHb-AIF and GBCA-AIF analyses results from the deconvolution of large similarly shaped AIF. This concern has been difficult to address as it would apply similarly to any injected contrast agent. Here we confirm that the similarity of hemodynamic measures using THx-dOHb-Step analysis, which is a direct measure, are similar to those derived from THx-dOHb-AIF and GBCA-AIF, which are model based, except for MTT as discussed.

2.4.2 Step [dOHb]

The Step re-oxygenation radically changes the pharmacokinetic considerations regarding the contrast. With the deconvolution-AIF process, data undergoes considerable averaging, resulting in poor resolution of small differences in measures. On the contrary, with THx-dOHb-Step, the abrupt change in [dOHb] of the blood at the lung is propagated to the MCA and all downstream cerebral vessels (FIG. 19). The increased duration for signal change in a voxel, which produces an increase in MTT, is not due to dispersion of the step change in capillary vessels but reflects the coursing of the contrast through the voxel. We would expect that scanning at a greater field strength and shorter TR would provide an increased time resolution to better fit the Gompertz function.

FIG. 19 illustrates the extent and repeatability of an abrupt step change in lung SaO₂ propagated to the middle cerebral artery. Three gas challenges 1901, 1902, 1903 were implemented in the subject. The gas challenge PO2 alternated between 40 mmHg and 95 mmHg; TR of 200 ms; time constants for reoxygenation for the gas challenges 1901, 1902, 1903 are 1.21 s, 1.67 s and 2.10 s, respectively.

### 3 Methods

### 3.1 Subjects and Ethics Approval

This study conformed to the standards set by the latest revision of the Declaration of Helsinki and was approved by the Research Ethics Board of the University Health Network (UHN) and Health Canada. Written informed consent to partake in this study was obtained from all subjects. We recruited 7 healthy individuals (5 M) between the ages of 24 and 60 (mean±SD=34.4±16.0 y) by word of mouth. They were non-smokers, not on any medication, and had no known history of neurological or cardiovascular disease. Exclusion criteria consisted of the presence of risk factors for the MRI environment.

### 3.2 Application of Contrast Agents

The standard sequence of changes in SaO₂ and [dOHb] used for these and other experiments were achieved by controlling the end-tidal partial pressures of oxygen (P_{ET}O₂) and carbon dioxide (P_{ET}CO₂) using the sequential delivery of inspired gases from a computer-controlled gas blender (RespirAct™; Thornhill Medical Inc, Toronto, Canada) running a prospective targeting algorithm. The principles of operation of the RespirAct™ have been previously described. With this targeting approach, the end tidal values have been shown to be equal, within measurement error, to their respective arterial partial pressures. Reduction in pulmonary PO2 is achieved by inhaling successive tidal volumes of prospectively blended hypoxic gas to dilute the oxygen in the functional residual capacity to target a P_{ET}O₂ of 40 mmHg. The re-oxygenation step targets the previous baseline P_{ET}O₂ of 95 mmHg. We avoid surpassing the previous baseline P_{ET}O₂ on re-oxygenation to facilitate rapid reinduction of similar hypoxia-reoxygenation steps.

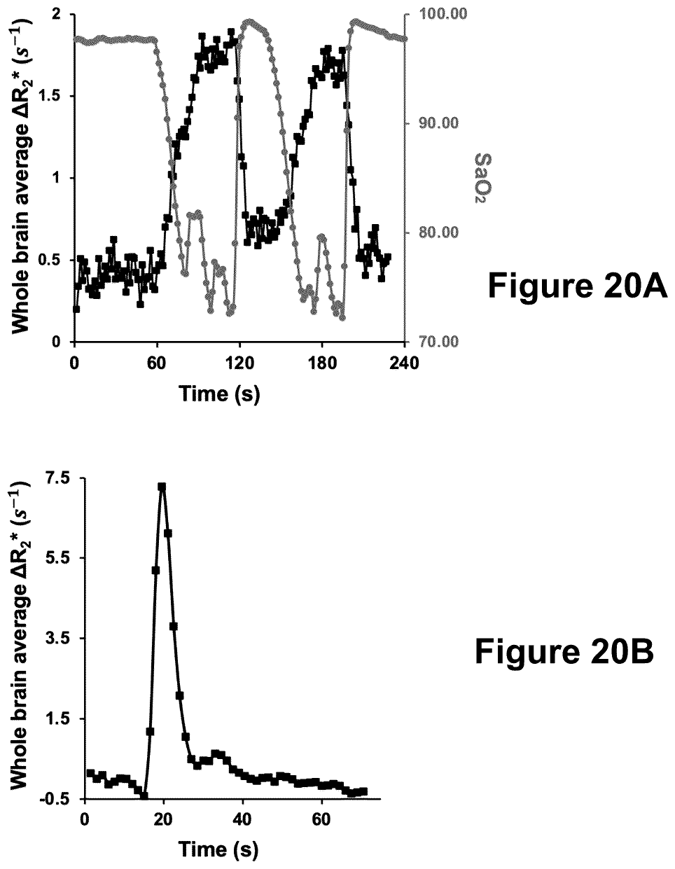

Subjects breathed through a facemask sealed to the face with skin tape (Tegaderm, 3M, Saint Paul, MN, U.S.A.) to exclude all but system-supplied gas. The programmed P_{ET}O₂ stimulus consisted of 60 s at 95 mmHg (normoxia), followed by 60 s at 40 mmHg (hypoxia), 20 s at normoxia, followed by 60 s hypoxia and return to normoxia for 60 s (FIG. 20A). P_{ET}CO₂ was held constant at the individual's resting value. The entire protocol was used for the THx-dOHb-AIF analysis, but only the terminal step increase in SaO₂ was used for the THx-dOHb-Step analysis. SaO₂ was calculated from the measured P_{ET}O₂ and P_{ET}CO₂, using the oxyhemoglobin dissociation curve relationship described previously in Equation 2, assuming a normal pH of 7.4 and a hemoglobin concentration of 130 g/L. After the completion of the P_{ET}O₂ targeting protocol, the subject returned to free breathing of room air for at least 5 min before an intravenous injection of 5 ml at 5 ml/s of Gadovist™ (Bayer, Canada), with a baseline delay of 20 s prior to injection, and flushed with 30 ml of saline to be used for the GBCA AIF analysis (FIG. 20B).

FIGS. 20A and 20B illustrate ΔR₂* signal waveforms for a representative subject in a voxel over the middle cerebral artery (MCA). FIG. 20A shows an example of data used for the THx-dOHb-AIF analysis showing the hypoxia-induced changes in SaO₂ (%) (red circles) and the AIF ΔR₂* signal (black squares) in a representative subject. FIG. 20B shows an example of data used for the GBCA AIF analysis showing the AIF ΔR₂* signal in a representative subject.

### 3.3 MRI Scanning Protocol

The experiments were performed in a 3-Tesla scanner (HDx Signa platform, GE healthcare, Milwaukee, WI, USA) with an 8-channel head coil. The scanning protocol consisted of a high-resolution T1-weighted scan followed by two T2*-weighted acquisitions. The high-resolution T1 scan was acquired using a 3D inversion prepared spoiled fast gradient echo sequence with the following parameters: T1=450 ms, TR 7.88 ms, TE=3 ms, flip angle=12°, voxel size=0.859×0.859×1 mm, matrix size=256×256, 146 slices, field of view=220 mm, no interslice gap. Both THx-dOHb and GBCA data acquisitions used a T2*-weighted gradient echoplanar imaging sequence with the following parameters: TR=1500 ms, TE=30 ms, flip angle=73°, 29 slices voxel size=3 mm isotropic with 64×64 matrix.

### 3.4 Data Analysis

The acquired T2*-weighted signal images were volume-registered, slice-time corrected and co-registered to the anatomical images using AFNI software (National Institutes of Health, Bethesda, Maryland, Version AFNI_24.0.12 ‘Caracalla’ URL https://afni.nimh.nih.gov). The acquisitions obtained during both THx-dOHb and GBCA were pre-processed in an identical manner to ensure no bias towards any one scan. The ‘spikes’ from the dataset were removed and a spatial blur of 5 mm was applied to each dataset using AFNI software. The T2*-weighted signal S (t) acquired during THx-dOHb and GBCA were converted to tissue concentration (ΔR₂*) using Equation 3.

First, the images from the THx-dOHb and GBCA acquisitions were analyzed using a conventional kinetic model-based approach. The visibly sharpest signal change over a middle cerebral artery was selected as the AIF and a deconvolution-based kinetic model of the type shown in FIG. 3A was used to calculate voxel-wise maps of MTT and rCBV. Standard tracer kinetic modeling was used to calculate MTT and rCBV as stated in Equation 5:

$$Δ {R}_{2}^{*} ( r ) = ( \frac{rCBV}{MTT} ) \times AIF ( t ) ⊗ R ( t ) + β 1 \times t + β 2 + ε ( t ) \qquad ( 5 )$$

$$where :$$

$$t = time$$

$$β1 and β2 account for linear signal drift and baseline t respectively$$

$$ε ( t ) represents the residuals .$$

$$R ( t ) = {e}^{- ( 1 / MTT )} the residue function ( FIG . 3 A )$$

The residue function was set equal to 1 at time 0 and 0 at time equal to 5×MTT and bound between 1 and 8 s. Metrics rCBV and MTT were determined using a least square fitting procedure. rCBF was then calculated as the ratio rCBV/MTT using the central volume theorem (FIG. 3A). Values of rCBV and rCBF were respectively multiplied by 10 and 50 to obtain easily readable values within the range of absolutes measures.

Second, the voxel-wise analysis of the ΔR₂* signal during the THx-dOHb step protocol was implemented using a custom analysis program (LabVIEW, National Instruments, Texas) as illustrated in FIG. 8. As FIG. 19 illustrates, the step change in arterial PO2, via reoxygenation from a hypoxic P_{ET}O₂ of approximately 40 mmHg to 95 mmHg, produces a step increase in SaO₂, and consequently a step decrease in [dOHb]. The ΔR₂* signal in a voxel decreases as blood as the increased SaO₂ displaces that at the hypoxic SaO₂. A reference cursor is placed by eye (a in FIG. 7) where the whole brain average ΔR₂* signal begins to decrease in response to the step increase in SaO₂ and acts as a time reference for all voxels for calculating relative arrival time rBAT.

The THx-dOHb Step analysis, explained in FIG. 7, proceeds as follows: For each voxel, a selected portion of the ΔR₂* signal response (FIG. 7, dots in black squares) before and after the reference cursor (a in FIG. 7) is fitted with the Gompertz fit function (FIG. 7, solid line) specified in Equation 4 using the Levenburg-Marquardt algorithm (National Instruments, Texas, LabVIEW), with R-squared indicating the goodness of fit. Fitting a function to the observed ΔR₂* signal serves to overcome the inherent noise, and a Gompertz function is used to describe the observed ΔR₂* signal response to the step change in SaO₂.

In FIG. 7, the squares are the ΔR₂* signal sampled at TR, with the Gompertz function Sfit (t) shown as a solid line. The dashed line slope is rCBF and its time range is MTT. The amplitude of change represents the rCBV. The geometry of the right-angle triangle formed by rCBF, MTT and rCBV verifies the Central Volume Theorem. Further explanation is given in the accompanying text.

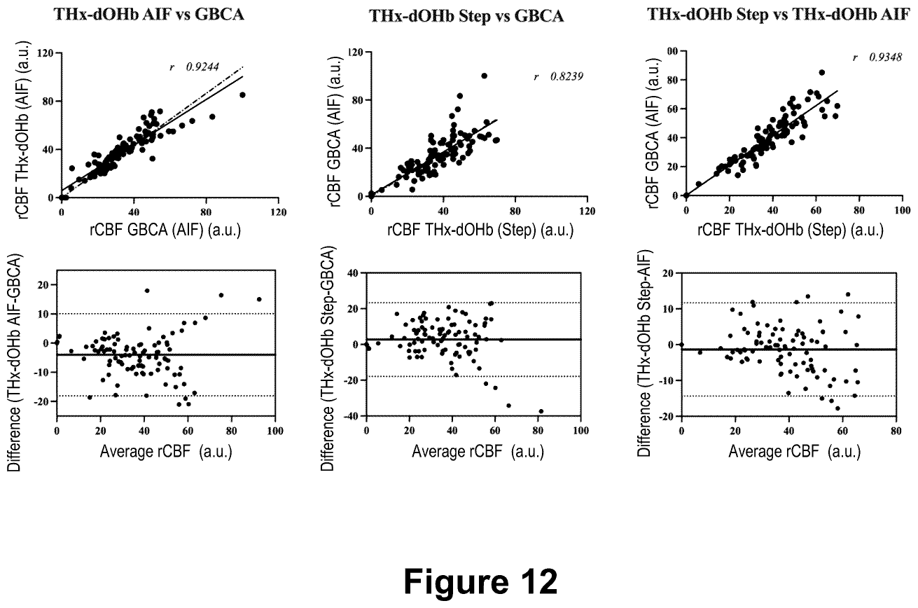

Perfusion metrics rCBV, rCBF and rBAT are all calculated independently as shown in FIG. 7 from the Gompertz function fit to the ΔR₂* signal step response, Sfit (t). MTT is the time range of the line fit (blue dashed line in FIG. 7), which equals rCBV/rCBF. Gompertz function fit parameter “a” measures the complete decrease in the ΔR₂* step response to calculate rCBV. The start of the ΔR₂* decrease (FIG. 12, line “b”) identifies the time where Sfit (t) begins to decrease by 2% of rCBV. Relative blood arrival time, rBAT, is calculated as the difference of start time (b)-reference time (a), with negative values signifying earlier arrival.

The maximum rate of decrease of the ΔR₂* signal step response is calculated from the Sfit (t) parameters as “a×c/e” to measure rCBF, where e is the base of natural logarithms. A line with this slope is drawn through the time of maximum slope, “In (b)/c” (FIG. 7, dashed line). It defines three temporal regions, as indicated by the arrows in FIG. 7. First, the exponential increase in the rate of decline of the ΔR₂* signal as the step change in SaO₂ arrives at the voxel until the change has entered the voxel in all capillaries; second, a linear portion of the ΔR₂* signal decline as all vessels fill with the change in SaO₂ until the change begins to leave the voxel; third, an exponential decay in the rate of decline of the ΔR₂* signal as the SaO₂ change leaves the voxel. MTT is the sum of the time constants of the first and third temporal regions plus the time taken in the second linear ΔR₂* signal decrease temporal region. Consequently, MTT satisfies the central volume theorem as the ratio of CBV/CBF. Values of rCBV and rCBF were respectively multiplied by 2 and 200 to obtain easily readable values within the range of absolutes measures.

The perfusion maps obtained from each analysis were transformed into Montreal Neurological Institute (MNI) space and overlayed onto their respective anatomical images. Analytical processing software, SPM8 (Wellcome Department of Imaging Neuroscience, Institute of Neurology, University College, London, UK, URL https://www.fil.ion.ucl.ac.uk/spm/software/spm8/), was used to segment the anatomical images (T1 weighted) into whole brain cortical supratentorial gray matter (GM) and white matter (WM). Average resting perfusion metrics using all three analyses were calculated for all subjects. The MTT, rCBF and rCBV maps for each healthy subject were compiled together to establish average normative ranges for each of the three analyses. This compilation was performed for each metric and analysis by calculating a voxel-by-voxel mean and standard deviations from the co-registered maps in standard space. Additional segmentation into 100 regions of interest (ROIs) was performed by coregistration of T1 images into the Talairach-Tournoux (TT) atlas. Using the TT atlas, ROI between pairs of perfusion metrics was calculated and plotted as scatterplots.

### 3.4 Statistical Analysis

Numerical comparisons of the perfusion metrics between the three analyses were not possible for relative values of rCBV and rCBF expressed in arbitrary units. MTT was compared using a two-way ANOVA with factors type of analysis and region (GM vs. WM). To assess the spatial contrast of the maps, we compared the GM-to-WM ratios using a one-way ANOVA. Both Normality Tests (Shapiro-Wilk) and Equal Variance Tests (Brown-Forsythe) were part of the ANOVA, and correction for multiple comparisons were applied by an all pairwise multiple comparison procedure (Holm-Sidak method). Significant difference for these tests was taken as P<0.05.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. A method of determining a perfusion metric in a subject comprising:
inducing a stepwise increase in arterial partial pressure of oxygen in a subject using a respiratory device to:
deliver a hypoxic gas to induce hypoxia in the subject's arterial blood; and then
deliver an oxygenated gas to reoxygenate the subject's arterial blood;
measuring a magnetic signal in a selected voxel using a magnetic resonance imaging system to derive a change in effective transverse relaxation rate (ΔR₂*) time course responsive to the stepwise increase in arterial partial pressure of oxygen; and
based on the ΔR₂* time course, computing a perfusion metric for the selected voxel.

2. The method of claim 1 wherein the hypoxic gas is delivered to the subject in successive tidal volumes over a series of breaths, and wherein the oxygenated gas is delivered to the subject within a single breath.

3. The method of claim 2 wherein the respiratory device is a sequential gas delivery apparatus, delivering the hypoxic gas includes targeting a first P_{ET}O₂ using the sequential gas delivery device, and delivering the oxygenated gas includes targeting a second P_{ET}O₂ higher than the first P_{ET}O₂ using the sequential gas delivery device; the method further comprising:
maintaining an end tidal partial pressure of carbon dioxide (P_{ET}CO₂) using the sequential gas delivery device while inducing the stepwise increase in arterial partial pressure of oxygen.

4. The method of claim 3 wherein the first P_{ET}O₂ is approximately 40 mmHg and the second P_{ET}O₂ is approximately 95 mmHg.

5. The method of claim 2 further comprising fitting a predetermined sigmoid function to the ΔR₂* time course, wherein computing the perfusion metric for the selected voxel is further based on the predetermined sigmoid function.

6. The method of claim 5 wherein the predetermined sigmoid function is a Gompertz function computed as follows:
$${S}_{fit} ( t ) = {S}_{base} + a {e}^{- {be}^{- ct}} ;$$
$$where :$$
$$S = Δ {R}_{2}^{*} ;$$
$$t = time ;$$
$${S}_{fit ( t )} = the fitted Δ {R}_{2}^{*} signal time course of the step response ;$$
$${S}_{base} = the initial value of {S}_{fit ( t )} ;$$
$$a = the magnitude of the S decrease ;$$
$$b = the displacement along the time axis ;$$
$$and$$
$$c = the rate of change .$$

7. The method of claim 6 wherein the hemoglobin concentration is measured or assumed to be approximately 130 g/L in healthy women and 150 g/L in healthy men.

8. The method of claim 6 wherein the pH is assumed to be about 7.4.

9. The method of claim 5 wherein the perfusion metric includes relative cerebral blood volume (rCBV) and computing the perfusion metric comprises computing the magnitude of the predetermined sigmoid function.

10. The method of claim 9 wherein the perfusion metric includes relative cerebral blood flow (rCBF) and computing the perfusion metric comprises computing the maximum rate of decrease in the predetermined sigmoid function.

11. The method of claim 10 wherein the perfusion metric includes mean transit time (MTT) and the perfusion metric is calculated as MTT=rCBV/rCBF.

12. The method of claim 1 further comprising:
computing a plurality of perfusion metrics for a respective plurality of voxels;
co-registering the perfusion metrics to an anatomical image; and
generating a perfusion map for the respective plurality of voxels.

13. The method of claim 4 further comprising:
comparing the perfusion metric to a statistical value representing the perfusion metric for corresponding voxels in a reference population; and
generating a z-score based on the comparison, the z-score representing the perfusion metric for the selected voxel relative to the reference population.

14. The method of claim 13 further comprising assessing or diagnosing a health condition based on the z-score.

15. The method of claim 14 wherein the health condition is a cardiovascular disease or neurological disease selected from: Parkinson's disease, stroke, hemangiomas, vascular tumor or cyst, coronary heart disease, Moyamoya disease, Cerebral Venous Thrombosis, Arteriovenous Malformation, arterio-venous fistulas, angioma formation, carotid artery disease, intracranial hypertension, steno-occlusive disease, and kidney insufficiency.

16. The method of claim 13 further comprising assessing a treatment based on the z-score.

17. A system for quantifying a perfusion metric in a subject comprising:
a respiratory device configured to induce a stepwise increase in arterial partial pressure of oxygen in a subject by:
delivering to the subject a hypoxic gas suitable to induce hypoxia in the subject's arterial blood; and
delivering to the subject an oxygenated gas to reoxygenate the subject's arterial blood;
a magnetic resonance imaging device configured to measure a magnetic signal in a selected voxel and derive a change in relaxation rate (ΔR₂*) time course in the subject responsive to the increase in arterial partial pressure of oxygen;
a processor for computing a perfusion metric for the selected voxel based on the ΔR₂* time course.

18. The method of claim 17 further comprising:
computing a plurality of perfusion metrics for a respective plurality of voxels;
co-registering the perfusion metrics to an anatomical image; and
generating a perfusion map for the respective plurality of voxels.

19. The method of claim 17 further comprising:
comparing the perfusion metric to a statistical value representing the perfusion metric for corresponding voxels in a reference population; and
generating a z-score based on the comparison, the z-score representing the perfusion metric for the selected voxel relative to the reference population.

20. The method of claim 19 further comprising assessing or diagnosing a health condition based on the z-score.
